(19) United States
(12) Patent Application Publication
(10) Pub. No.: US 2018/0008723 A1
(43) Pub. Date: Jan. 11, 2018

(54) MULTIMERIC COMPOUNDS OF A KRINGLE DOMAIN FROM THE HEPATOCYTE GROWTH FACTOR / SCATTER FACTOR (HGF/SF)

(71) Applicants: UNIVERSITE DES SCIENCES ET TECHNOLOGIES DE LILLE-LILLE 1, Villeneuve D'ascq (FR); CENTRE NATIONAL DE LA RECHERCHE SCIENTIFIQUE, Paris Cedex 16 (FR); INSTITUT PASTEUR DE LILLE, Lille Cedex (FR); UNIVERSITE DE LILLE 2 DROIT ET SANTE, Lille (FR); UNIVERSITA' DEGLI STUDI DI PAVIA, Pavia (IT)

(72) Inventors: Jerome VICOGNE, PERENCHIES (FR); Oleg MELNYK, ANNOEULLIN (FR); Nathalie OLLIVIER, ROUBAIX (FR); Eric ADRIAENSSENS, FAUMONT (FR); Berenice LECLERCQ, WASQUEHAL (FR); Claire SIMONNEAU, JAUNAY-CLAN (FR); Giovanni DE NOLA, PAVIA (IT); Ermanno GHERARDI, PAVIA (IT); Hugo DE JONGE, PAVIA (IT)

(21) Appl. No.: 15/545,163

(22) PCT Filed: Jan. 21, 2016
(86) PCT No.: PCT/EP2016/051268

§ 371 Date: Jul. 20, 2017

(30) Foreign Application Priority Data
Jan. 21, 2015 (EP) 15152029.3

(51) Int. Cl. G01N 33/58; A61K 38/18; A61K 49/00; G01N 33/50

(52) U.S. Cl. CPC A61K 47/665; G01N 33/582; G01N 33/502; A61K 47/557; A61K 38/1833; A61K 49/0002

(57) ABSTRACT

Disclosed are multimeric compounds of K1 domains from the Hepatocyte Growth Factor/Scatter Factor (HGF/SF) being able to induce activation of the tyrosine kinase receptor MET and their uses.

The present invention relates to multimeric compounds of K1 domains from the Hepatocyte Growth Factor/Scatter Factor (HGF/SF).

Hepatocyte growth factor/scatter factor (HGF/SF) is a secreted 90 kDa protein with a complex domain structure which is synthesised as an inactive precursor and is subsequently converted proteolytically to a two-chain (α/β) active species (Nakamura, T., Structure and function of hepatocyte growth factor. Prog Growth Factor Res 3, 67-85 (1991); Holmes et al., Insights into the structure/function of hepatocyte growth factor/scatter factor from studies with individual domains. J Mol Biol 367, 395-408 (2007)). The a chain consists of an N terminal domain (N) and four copies of the kringle domain (K1, K2, K3 and K4). The β chain is a catalytically inactive serine proteinase homology domain (SPH). Two receptor binding sites have been identified in HGF/SF: a high-affinity site located in the N-terminal region of the a chain and a low-affinity one located in the β chain.

HGF/SF is a potent growth and motility factor discovered independently as a liver mitogen (hepatocyte growth factor, HGF) (Miyazawa et al., Molecular cloning and sequence analysis of cDNA for human hepatocyte growth factor. Biochem Biophys Res Commun 163, 967-973 (1989); Nakamura et al., Purification and subunit structure of hepatocyte growth factor from rat platelets. FEBS Lett 224, 311-316 (1998); Zarnegar et al., Purification and biological characterization of human hepatopoietin a, a polypeptide growth factor for hepatocytes. Cancer Res 49, 3314-3320 (1989)) and a fibroblast-derived, epithelial motility factor (scatter factor, SF) (Stoker et al., Scatter factor is a fibroblast-derived modulator of epithelial cell mobility. Nature 327, 239-242 (1987); Gherardi et al., Purification of scatter factor, a fibroblast-derived basic protein that modulates epithelial interactions and movement. Proc Natl Acad Sci USA 86, 5844-5848 (1989)). A receptor tyrosine kinase MET encoded by a proto-oncogene was subsequently demonstrated to be the receptor for HGF/SF (Bottaro et al., Identification of the hepatocyte growth factor as the c-met proto-oncogene product. Science 251, 802-804 (1991)).

Interestingly, the primary HGF/SF transcript encodes two alternative splice variants. The first variant is caused by a premature translation termination and generates the NK1 protein containing the N domain and the first Kringle domain (K1) of HGF/SF. NK1 protein possesses a marked agonist activity but requires heparan sulphate interaction to induce complete MET activation. Structurally, NK1 protein consists of two globular domains that, in the presence of heparin, form a “head to tail” homodimer probably responsible for the MET dimerisation and activation (Chirgadze et al., Crystal structure of the NK1 fragment of HGF/SF suggests a novel mode for growth factor dimerization and receptor binding. Nat Struct Biol 6, 72-79 (1999). The second splice variant, also generated by a premature termination, produces the NK2 protein, containing the N domain and the first two kringle domains. NK2 is considered as a natural MET antagonist. Indeed, NK2 maintains its MET binding capacity, but due to its conformational properties, lacks the ability to activate MET. However, structure-based targeted mutations allow NK2 to be efficiently switched from MET antagonist to agonist by repositioning the K1 domain in a conformation close to that of NK1.

Beside many attempts to propose a unified and convergent interaction model, the MET binding mechanisms of HGF/SF are still unclear and controversial. In particular, no crystal structure of NK1 in complex with a soluble MET extracellular domain is yet available. HGF/SF is a bivalent ligand that contains a high and low affiny binding sites for MET located respectively in the N-terminal region of the α-chain (N and/or K1 domains) and in the β-chain (SPH domain). Binding of HGF/SF to the MET ectodomain in solution yields complexes with 2:2 stoichiometry (Gherardi et al., Structural basis of hepatocyte growth factor/scatter factor and met signalling. Proc Natl Acad Sci USA 103, 4046-4051 (2006)). The SPH domain binds MET with a well-defined interface. However, the localisation of NK1 binding site on MET is still unclear, and the exact HGF/SF-MET interaction model remains controversial (Holmes et al., Insights into the structure/function of hepatocyte growth factor/scatter factor from studies with individual domains. J Mol Biol 367, 395-408 (2007); Stamos et al., Crystal structure of the HGF beta-chain in complex with the sema domain of the met receptor. The EMBO Journal 23, 2325-2335 (2004); Merkulova-Rainon et al., The N-terminal domain of hepatocyte growth factor inhibits the angiogenic behavior of endothelial cells independently from binding to the c-Met-receptor. J Biol Chem 278, 37400-37408 (2003)).

HGF/SF and MET play essential physiological roles both in development and in tissue/organ regeneration. In particular, HGF/SF-MET is essential for liver and skin regeneration after hepatectomy (Huh et al., Hepatocyte growth factor/c-met signaling pathway is required for efficient liver regeneration and repair. Proc Natl Acad Sci USA 101, 4477-4482 (2004); Borowiak et al., Met provides essential signals for liver regeneration. Proc Natl Acad Sci USA 101, 10608-10613 (2004)) and skin wounds (Chmielowiec et al., C-met is essential for wound healing in the skin. J Cell Biol 177, 151-162 (2007)). HGF/SF further protects cardiac and skeletal muscle from experimental damage (Urbanek et al., Cardiac stem cells possess growth factor-receptor systems that after activation regenerate the infarcted myocardium, improving ventricular function and long-term survival. Circ Res 97, 663-673 (2005)), delays progression of a transgenic model of motor neuron disease (Sun et al., Overexpression of HGF retards disease progression and prolongs life span in a transgenic mouse model of als. J Neurosci 22, 6537-6548 (2002)) and an immunological model of multiple sclerosis (Bai et al., Hepatocyte growth factor mediates mesenchymal stem cell-induced recovery in multiple sclerosis models. Nature neuroscience 15, 862-870 (2012)). Together, these studies highlight a vast potential of HGF/SF in regenerative medicine, a concept supported by a number of pre-clinical and more recent clinical studies. Moreover, this ligand-receptor pair is also frequently involved in tumorigenesis and metastasis processes, and therefore constitutes a major target for the development of cancer therapies.

In particular, many investigations are concentrating on HGF/SF agonist synthesis to allow tissue regeneration, especially for liver regeneration after hepatectomy or lesions involved in diabetes diseases.

Since the current knowledge of the HGF/SF-MET interactions does not allow the rational design of HGF/SF-MET signalling inhibitors or agonists, the usefulness of HGF/SH has been established using native HGF/SF, gene delivery methods and NK1-based MET agonists.

However, native HGF/SF is a protein with limited tissue diffusion reflecting its role as a locally-acting tissue morphogen (Birchmeier et al., Met, metastasis, mobility and more. Nat Rev Mol Cell Biol 4, 915-925 (2003); Ross et al., Protein Engineered Variants of Hepatocyte Growth Factor/Scatter Factor Promote Proliferation of Primary Human Hepatocytes and in Rodent Liver. Gastroenterology 142, 897-906 (2012)). Indeed, after its local or systemic administration, HGF/SF is immobilized by heparan sulphate present in the extracellular matrix, resulting in a severely decreased diffusion towards MET receptors in more distant tissues (Roos et al., Induction of liver growth in normal mice by infusion of hepatocyte growth factor/scatter factor. The American Journal of Physiology 268, G380-386 (1995); Hartmann et al., Engineered mutants of HGF/SF with reduced binding to heparan sulphate proteoglycans, decreased clearance and enhanced activity in vivo. Curr Biol 8, 125-134 (1998)). Moreover, native HGF/SH is also difficult and costly to produce owing to its complex, multidomain structure.

Gene delivery methods, including intramuscular injection of naked DNA encoding HGF/SF addresses several of the problems associated with the use of native HGF/SF as a protein therapeutic (the cost of production of the HGF/SF protein, for example). Clinical trials with HGF/SF DNA are currently conducted in patients with diabetic peripheral neuropathy and in patients with amyotrophic lateral sclerosis. The results of these studies are awaited with interest but there remain limitations with the current gene delivery methods in terms of the achievement of stable therapeutic levels of the gene products and the relative availability to specific tissue domains and organs due to limited diffusion. For example, such gene delivery methods are based on plasmid delivery systems (patent applications WO 2009/093880, WO 2009/125986 and WO 2013/065913) or adenovirus-based delivery systems (Yang et al., Improvement of heart function in postinfarct heart failure swine models after hepatocyte growth factor gene transfer: comparison of low-, medium- and high-doses groups. Mol Biol Rep 37, 2075-2081 (2010)).

Currently available NK1-based MET agonists, such as NK1 (i.e. a NK1 mutant), have a strong affinity and offer advantages over HGF/SF (Lietha et al., Crystal structures of NK1-heparin complexes reveal the basis for NK1 activity and enable engineering of potent agonists of the MET receptor. The EMBO journal 20, 5543-5555 (2001) and patent U.S. Pat. No. 7,179,786). Unlike native HGF/SF, this NK1 mutant can be effectively produced in heterologous expression systems, is stable in physiological buffers and thus can be administered with full control over dosage and plasma concentration. However, a potential limitation of NK1 is its strong residual affinity for heparan sulphate that avoids tissue diffusion.

Therefore, there is a need for potent MET agonists with an improved stability, an improved shelf life, an optimal bioavailability, and that can be produced at low cost and easily administered.

One of the aims of the invention is to provide a K1-based multimeric compound able to induce activation of the tyrosine kinase receptor MET.

Another aim of the invention is also to provide compositions containing said K1-based multimeric compound.

Another aim of the invention further relates to the use of said K1-based multimeric compound, in particular for diagnostical and therapeutical applications.

The present invention relates to a multimeric compound comprising at least two K1 peptide domains (Kringle 1) of the Hepatocyte Growth Factor/Scatter Factor (HGF/SF) and being represented by the formula (I):

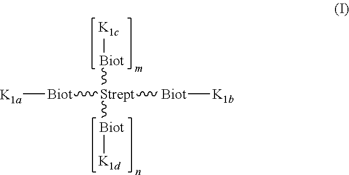

wherein:
m=0 or 1,
n=0 or 1,
K1ₐ, K1_{b}, and, if present, K1_{c} and K1_{d} are polypeptides,
K1ₐ and K1_{b} and, if present, K1_{c} and K1_{d} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule chosen among the group consisting of: streptavidin, avidin, neutravidin and any synthetic or recombinant derivatives thereof,
K1ₐ and K1_{b} and, if present, K1_{c} and K1_{d} are C-terminally linked to a Biot by a covalent bond, and each Biot is linked to Strept by a non-covalent bond,
[symbol] , [symbol] , [symbol] and [symbol] represent a non-covalent bond,
- and [symbol] represent a covalent bond,
   said multimeric compound being able to induce activation of the tyrosine kinase receptor MET.

The present application is based on the two-pronged observation made by the Inventors that K1 domain constitutes the building block for potent MET agonists and that the streptavidin technology allows to reconstitute a head-to-tail homodimer mimicking the active signaling conformation of K1 domains in the NK1 dimer.

The examination of the crystal structure of the NK1 homodimer shows a distance of about 2.3 nm between the two NK1 C-termini of HGF/SF, which is very close to the distance between two biotin binding sites on the same face of a streptavidin tetramer. Surprisingly, the K1-B streptavidin complex was found to be a potent MET agonist.

The multimeric compound of the invention has many technical and financial advantages. The most important technical advantage is that the multimeric compound of the invention has a potent MET agonistic activity. Thus, the multimeric compound is able to activate the MET receptor and/or induce any phenotype associated to the MET activation in various in vitro and in vivo assays.

The multimeric compound has a MET agonistic activity if it is able to:
bind the multimeric compound to the MET receptor,
activate the MET phosphorylation and the downstream signaling in cells, and
induce at least one cellular phenotype such as survival, proliferation, morphogenesis and/or migration.

The validation of these criteria can be shown using protein-protein interaction tests (such as SPR (Surface Plasmon Resonance), AlphaScreen, Pull-Down technique or gel-filtration chromatography), phosphorylation tests (such as western-blot, ELISA or AlphaScreen) and phenotypic tests (such as scattering, MTT assay or matrigel induced morphogenesis).

For example, MET activation and downstream signaling in cells can be analyzed in vitro by western blot and quantified by homogeneous time resolved fluorescence (HTRF) approaches. In vivo, it is also possible to analyze local angiogenesis and protection of mice from Fas-induced fulminant hepatitis.

Another technical advantage is that the multimeric compound of the invention is a protein complex which can be administered with full control over dosage and/or plasma concentration.

Moreover, one of the major disadvantages of native HGF/SF is the fact that it strongly binds to heparan sulphates in the extracellular matrix. This severely limits the diffusion of the molecule to more distant sites. The multimeric compound of the invention in contrast is missing the high affinity heparan sulphate site.

Therefore, the multimeric compound of the invention is not immobilized by heparan sulphate chains of extracellular matrix, contrary to HGF/SF. Therefore, when injected into a patient, the multimeric compound can diffuse from the area of injection towards MET receptors in distant tissues, whereas native HGF/SF is unable to do.

Some of the financial advantages are that the multimeric compound of the invention can be easily synthesized and obtained in large amounts.

The chemical synthesis gives a clean environment with no possibilities of contamination from the host cells commonly used as expression systems (such as bacteria or yeasts).

Moreover, the chemical synthesis gives a controlled environment to modulate the multimeric structure of the compound obtained, i.e. to obtain in particular dimeric, trimeric or and tetrameric compounds.

In the invention, the expressions “K1-B”, “K1B”, “K1-Biot” or “biotinylated version of the K1 domain” all refer to a biotinylated peptide comprising or consisting of the sequence of a K1 domain of HGF/SF.

In the invention, the expressions “multimeric compound comprising at least two K1 peptide domains”, “K1B/S complex” or “K1-B streptavidin complex” refer to a molecular complex which comprises at least two biotinylated versions of the K1 domain of HGF/SF each linked to the same molecule of streptavidin, avidin, neutravidin or any synthetic or recombinant derivatives thereof, by a non-covalent bond.

In the invention, streptavidin, avidin, neutravidin, or any synthetic or recombinant derivatives thereof, are preferentially used under a tetravalent form, but can also be used under trivalent or bivalent forms.

In one embodiment, the invention relates to a multimeric compound, wherein Strept represents one molecule of streptavidin.

In one embodiment, the invention relates to a multimeric compound, wherein Strept represents one molecule of avidin.

In the formula (I), [symbol] , [symbol] , [symbol] and [symbol] represent a non-covalent bond.

In the formula (I), - and [symbol] represent a covalent bond.

In the formula (I), if m=1 and n=1, the multimeric compound contains 4 K1-Biot and thus, is a tetramer of K1 domains.

In the formula (I), if m=1 and n=0, or if m=0 and n=1, the multimeric compound contains 3 K1-Biot and thus, is a trimer of K1 domains.

In the formula (I), if m=0 and n=0, the multimeric compound contains 2 K1-Biot and thus, is a dimer of K1 domains.

In one embodiment, the invention relates to a multimeric compound, said multimeric compound being a dimer containing two K1 peptide domains.

In one embodiment, the invention relates to a multimeric compound, said multimeric compound being a trimer containing three K1 peptide domains.

In one embodiment, the invention relates to a multimeric compound, said multimeric compound being a tetramer containing four K1 peptide domains.

In one embodiment, the invention relates to a multimeric compound, which is a K1 dimer represented by the formula (II):

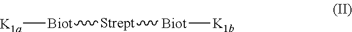

wherein:
K1ₐ and K1_{b} are polypeptides,
K1ₐ and K1_{b} contain a K1 peptide domain, said K1 peptide domain consisting of amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ and K1_{b} are C-terminally linked to a Biot by a covalent bond, and each Biot is linked to Strept by a non-covalent bond.

In one embodiment, the invention relates to a multimeric compound, which is a K1 trimer represented by the formula (III):

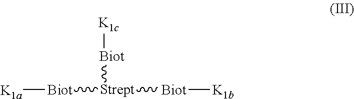

wherein:
K1ₐ, K1_{b} and K1, are polypeptides,
K1ₐ, K1_{b} and K1, contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b} and K1, are C-terminally linked to a Biot by a covalent bond, and each Biot is linked to the Strept by a non-covalent bond.

In one embodiment, the invention relates to a multimeric compound, which is a K1 tetramer represented by the formula (IV):

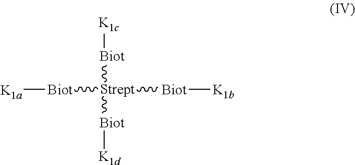

wherein:
K1ₐ, K1_{b}, K1_{c} and K1_{d} are polypeptides,
K1ₐ, K1_{b}, K1_{c} and K1_{d} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b}, K1, and K1_{d} are C-terminally linked to a Biot by a covalent bond, and each Biot is linked to Strept by a non-covalent bond.

In the invention, K1ₐ, K1_{b}, K1_{c} and K1_{d} are polypeptides that contain a K1 domain, thus they comprise or, preferably, consists of a K1 domain.

SEQ ID NO: 1 corresponds to the sequence of the human K1 domain, i.e. the region from the amino acid in position 128 to the amino acid position 206 of HGF/SF represented by SEQ ID NO: 3.

SEQ ID NO: 1 has a size of 79 amino acids and is flanked by two cysteines.

SEQ ID NO: 2 corresponds to the variant of the human K1 domain in which 5 amino acids are missing.

TABLE 1
Sequence of the K1 domain and its variant.

| | |
|---|---|
| SEQ ID NO: 1 | CIIGKGRSYKGTVSITKSGIKCQPWSSMIPHEHSFLP |
| | SSYRGKDLQENYCRNPRGEEGGPWCFTSNPEVRYEVC |
| | DIPQC |
| SEQ ID NO: 2 | CIIGKGRSYKGTVSITKSGIKCQPWSSMIPHEHSYRG |
| | KDLQENYCRNPRGEEGGPWCFTSNPEVRYEVCDIPQC |
| SEQ ID NO: 3 | MWVTKLLPALLLQHVLLHLLLLPIAIPYAEGQRKRRN |
| | TIHEFKKSAKTTLIKIDPALKIKTKKVNTADQCANRC |
| | TRNKGLPFTCKAFVFDKARKQCLWFPFNSMSSGVKKE |
| | FGHEFDLYENKDYIRNCIIGKGRSYKGTVSITKSGIK |
| | CQPWSSMIPHEHSFLPSSYRGKDLQENYCRNPRGEEG |
| | GPWCFTSNPEVRYEVCDIPQCSEVECMTCNGESYRGL |
| | MDHTESGKICQRWDHQTPHRHKFLPERYPDKGFDDNY |
| | CRNPDGQPRPWCYTLDPHTRWEYCAIKTCADNTMNDT |
| | DVPLETTECIQGQGEGYRGTVNTIWNGIPCQRWDSQY |
| | PHEHDMTPENFKCKDLRENYCRNPDGSESPWCFTTDP |
| | NIRVGYCSQIPNCDMSHGQDCYRGNGKNYMGNLSQTR |
| | SGLTCSMWDKNMEDLHRHIFWEPDASKLNENYCRNPD |
| | DDAHGPWCYTGNPLIPWDYCPISRCEGDTTPTIVNLD |
| | HPVISCAKTKQLRVVNGIPTRTNIGWMVSLRYRNKHI |
| | CGGSLIKESWVLTARQCFPSRDLKDYEAWLGIHDVHG |
| | RGDEKCKQVLNVSQLVYGPEGSDLVLMKLARPAVLDD |
| | FVSTIDLPNYGCTIPEKTSCSVYGWGYTGLINYDGLL |
| | RVAHLYIMGNEKCSQHHRGKVTLNESEICAGAEKIGS |
| | GPCEGDYGGPLVCEQHKMRMVLGVIVPGRGCAIPNRP |
| | GIFVRVAYYAKWIHKIILTYKVPQS |

The variant of the human K1 domain, represented by SEQ ID NO: 2, differs from the human K1 domain, represented by SEQ ID NO: 1, by a deletion of 5 consecutive amino acids: SFLPS.

In the invention, K1ₐ, K1_{b}, K1_{c} and K1_{d} contain a K1 peptide domain. Said K1 peptide domain can consist of an amino acid sequence SEQ ID NO: 1 or of an amino acid sequence with at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1.

The “percentage identity” between two peptide sequences, as defined in the present invention, is determined by comparing two sequences aligned optimally, through a window of comparison. The amino acid sequence in the comparison window may thus comprise additions or deletions (e.g. “gaps”) relative to the reference sequence (which does not include these additions or deletions) so to obtain an optimal alignment between the two sequences.

The percentage identity is calculated by determining the number of positions in which an amino acid residue is identical in the two compared sequences and dividing this number by the total number of positions in the window of comparison and multiplying the result by one hundred to obtain the percent identity of two amino acid sequences to each other.

The percentage identity may be determined over the entire amino acid sequence or over selected domains, preferably over the entire amino acid sequence. For local alignments, the Smith-Waterman algorithm is particularly useful (Smith T F, Waterman M S (1981) J. Mol. Biol. 147(1); 195-7).

In one embodiment, the invention relates to a multimeric compound, wherein K1ₐ and K1_{b} are identical.

In one embodiment, the invention relates to a multimeric compound, wherein K1ₐ, K1_{b} and K1_{c} are identical.

In one embodiment, the invention relates to a multimeric compound, wherein K1ₐ, K1_{b}, K1_{c} and K1_{d} are identical.

In one embodiment, the invention relates to a multimeric compound, wherein K1ₐ, K1_{b}, K1_{c} and K1_{d} are all different from each other.

In one embodiment, the invention relates to a multimeric compound, wherein K1ₐ, K1_{b}, K1_{c} and K1_{d} consist of an amino acid sequence SEQ ID NO: 1.

In one embodiment, the invention relates to a multimeric compound, wherein K1ₐ, K1_{b}, K1_{c} and K1_{d} consist of an amino acid sequence SEQ ID NO: 2.

In the invention, the size of the polypeptides K1ₐ, K1_{b}, K1_{c} and K1_{d} is at least 70 amino acids.

The size of the K1 peptide domain is at least 70 amino acids, preferably at least 74 amino acids, more preferably at least 79 amino acids.

In particular, the size of the K1 peptide domain is 70 to 100 amino acids. Such a K1 peptide domain can consist of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 amino acids.

In one embodiment, the invention relates to a multimeric compound, wherein said activation of the tyrosine kinase receptor MET is heparan sulfate independent.

In in vivo conditions, HGF/SF is immobilized by heparan sulphate chains present in the extracellular matrix, resulting in a severely reduced diffusion and/or tissue distribution.

The protein of the invention is missing the high affinity heparan sulphate binding site (N domain) and therefore is able to diffuse towards MET receptors in distant tissues.

In one embodiment, the invention relates to a multimeric compound which is able to bind the tyrosine kinase receptor MET with a dissociation constant K_{D}≦200 nM, preferably ≦100 nM, more preferably ≦10 nM.

In particular, said multimeric compound is able to bind the tyrosine kinase receptor MET with a dissociation constant K_{D}≦200 nM, ≦150 nM, ≦100 nM, ≦90 nM, ≦80 nM, ≦70 nM, ≦60 nM, ≦50 nM, ≦40 nM, ≦30 nM, ≦10 nM, or ≦5 nM.

In one embodiment, the invention relates to a multimeric compound, wherein the distance between the C-termini of said at least two K1 peptide domains is 1.3-3.5 nm, preferably 2.0 to 2.3 nm.

In particular, the distance between the C-termini of said at least two K1 peptide domains is 1.3 nm, 1.4 nm, 1.5 nm, 1.6 nm, 1.7 nm, 1.8 nm, 1.9 nm, 2.0 nm, 2.1 nm, 2.2 nm, 2.3 nm, 2.4 nm, 2.5 nm, 2.6 nm, 2.7 nm, 2.8 nm, 2.9 nm, 3.0, nm 3.1 nm, 3.2 nm, 3.3 nm, 3.4 nm or 3.5 nm.

In another aspect, the invention relates to a process to obtain a multimeric compound comprising at least two K1 peptide domains, comprising the steps of:
synthesizing a molecule containing a K1 peptide domain linked to a biotin to obtain a biotinylated K1 molecule, said biotin being linked to the C-terminus of the K1 molecule,
mixing said biotinylated K1 molecule with a streptavidin homotetramer to obtain a composition of a multimeric compound comprising at least 2 K1 peptide domains,
purifying and separating multimeric compounds to obtain dimeric compounds of K1 domains, trimeric compounds of K1 domains, and tetrameric compounds of K1 domains.

The chemical synthesis of the multimeric compounds of the invention provides the advantage to eliminate any trace of bacterial or yeast contamination in comparison of NK1-based MET agonists produced in heterologous expression.

In an embodiment, the chemical synthesis of the K1 peptide domain linked to a biotin is performed using solid phase peptide synthesis (SPPS) in a one-pot sequential peptide segments assembly process, preferably a one-pot sequential three peptide segments assembly process.

The one-pot sequential peptide segments assembly process is a strategy whereby peptide segments are subjected to successive chemical reactions in just one reactor, avoiding a lengthy separation process and purification of the intermediate chemical compounds. For example, three segments of a K1 domain, segment 1, segment 2 and segment 3 are prepared, the latter containing a biotin extension. Segment 1 and 2 are joined together, and then, segment 1-2 is joined with segment 3 biotinylated to obtain a biotinylated K1 molecule.

In another aspect, the invention relates to a process to obtain a composition comprising a multimeric compound comprising at least two K1 peptide domains, comprising the steps of:
synthesizing a molecule containing a K1 peptide domain linked to a biotin to obtain a biotinylated K1 molecule, said biotin being linked to the C-terminus of the K1 molecule,
mixing said biotinylated K1 molecule with a streptavidin homotetramer with to obtain a composition of a multimeric compound comprising at least 2 K1 peptide domains, said biotinylated K1 molecule and said streptavidin homotetramer (K1B:S) being preferably mixed in a 2:1 molar ratio to obtain dimeric compounds of K1 domains, a 3:1 molar ratio to obtain trimeric compounds of K1 domains, or a 4:1 molar ratio to obtain tetrameric compounds of K1 domains.

In particular, said biotinylated K1 molecule and said streptavidin homotetramer are preferably mixed in a molar ratio from 2:1 to 8:1.

Dimeric, trimeric, and/or tetrameric compounds of K1 domains can be identified by SDS-PAGE analysis and by mass spectrometry analysis.

In another aspect, the invention also relates to a composition comprising a multimeric compound as defined above.

In one embodiment, the invention relates to a composition wherein said multimeric compound is in the form of a mix of:

a K1 dimer represented by the formula (II),

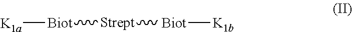

wherein:
   K1ₐ and K1_{b} are polypeptides,
   K1ₐ and K1_{b} contain a K1 peptide domain, said K1 peptide domain consisting of an amino acid sequence SEQ ID NO: 1 or of an amino acid sequence with at least 80%, preferably 90% identity to SEQ ID NO: 1,
   Biot represents one molecule of biotin, and Strept represents one molecule of streptavidin,
   K1ₐ and K1_{b} are C-terminally linked to Biot by a covalent bond, and each Biot is linked to Strept by a non-covalent bond,

a K1 trimer represented by the formula (III),

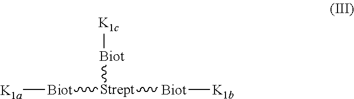

wherein:
K1ₐ, K1_{b} and K1_{c} are polypeptides,
K1ₐ, K1_{b} and K1_{c} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b} and K1_{c} are C-terminally linked to Biot by a covalent bond, and each Biot is linked to Strept by a non-covalent bond,
   and, a K1 tetramer represented by the formula (IV),

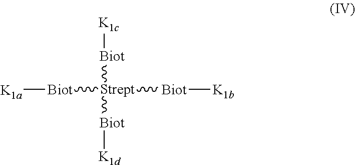

wherein:
K1ₐ, K1_{b}, K1_{c} and K1_{d} are polypeptides,
K1ₐ, K1_{b}, K1_{c} and K1_{d} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b}, K1_{c} and K1_{d} are C-terminally linked to a Biot by a covalent bond, and each Biot is linked to Biot by a non-covalent bond.

In one embodiment, the invention relates to a composition as defined above wherein at least 10% of said multimeric compound is in the form of a K1 dimer, preferably at least 70%, more preferably at least 90%.

In particular, the invention relates to a composition as defined above wherein at least 10%, 15%, 20%, 25%, 30%, 35%, 40%, 45%, 50%, 55%, 60%, 65%, 70%, 75%, 80%, 85%, 90% or 95% of said multimeric compound is in the form of a K1 dimer.

In another aspect, the invention relates to the use of a multimeric compound as defined above, as an in vitro diagnostic tool.

Due to its potent MET agonistic activity, the multimeric compound of the invention can be used to understand the mechanism of interaction between MET and HGF/SF.

In another aspect, the invention also relates to a multimeric compound as defined above, for use in an in vivo diagnostic method.

Due to its capacity to bind MET the multimeric compound of the invention represents a valuable tool for diagnostic methods, in particular for pathologies which implicate expression of HGF/SF and MET molecules.

In one embodiment, the invention relates to a multimeric compound, for use in an in vivo diagnostic method of a pathology chosen among: cancers, diseases of epithelial organs including acute and chronic liver diseases, acute and chronic kidney diseases, chronic lung diseases and chronic skin wounds, diseases of the central nervous system including neuron diseases and sclerosis, ischemic heart diseases, peripheral vascular diseases, diabetes and associated complications such as peripheral neuropathies.

In one embodiment, the invention relates to a multimeric compound, for use in an in vivo diagnostic method as defined above, wherein said cancers are tumors expressing the tyrosine kinase receptor MET.

In another aspect, the invention also relates to a multimeric compound, for use in medical imaging.

In another aspect, the invention also relates to a multimeric compound, for use in in vivo imaging.

Indeed, the multimeric compound of the invention can be labelled with a marker and allows the detection, localization and quantification of MET receptors.

For example, the multimeric compound can be labelled with radiopharmaceutical tracers or fluorescent tracers.

Such radiopharmaceutical tracers include, but are not limited to, Calcium-47, Carbon-11, Carbon-14, Chromium-51, Cobalt-57, Cobalt-58, Erbium-169, Fluorine-18, Gallium-67, Gallium-68, Hydrogen-3, Indium-111, Iodine-123, Iodine-125, Iodine-131, Iron-59, Krypton-81m, Nitrogen-13, Oxygen-15, Phosphorus-32, Radium-223, Rubidium-82, Samarium-153, Selenium-75, Sodium-22, Sodium-24, Strontium-89, Technetium-99m, Thallium-201, Xenon-133 and Yttrium-90.

Such fluorescent tracers include, but are not limited to, fluorescent dyes (such as rhodamine derivatives, coumarin derivatives, fluorescein derivatives, . . . ) or fluorescent proteins (such as GFP (green), YFP (yellow), RFP (red) . . . ).

In particular, infrared (IR) and near infrared (NIR) dyes and fluorescent proteins are preferred tracers for in vivo imaging due to increased penetration and reduced autofluorescence.

In one embodiment, the invention also relates to a multimeric compound for use in medical imaging as defined above, wherein said multimeric compound allows the detection and/or the tracking of drugs and/or imaging agent.

In particular, the multimeric compound of the invention can be used in image guided surgery. Pre- and intra-operative imaging is currently used to assist surgeons in the careful positioning of surgical tools as well as guiding the complete removal of specific tissue. Fluorescent (IR/NIR) probes may be used for live imaging during operation.

In another aspect, the invention also relates to the use of a multimeric compound as defined above, for the in vitro diagnostic of a pathology, said pathology being chosen among: cancers, diseases of epithelial organs including acute and chronic liver diseases, acute and chronic kidney diseases, chronic lung diseases and chronic skin wounds, diseases of the central nervous system including neuron diseases and sclerosis, ischemic heart diseases, peripheral vascular diseases, diabetes and associated complications such as peripheral neuropathies.

In one embodiment, the invention relates to the use of a multimeric compound, for the in vitro diagnostic of cancers, wherein said cancers are tumors expressing the tyrosine kinase receptor MET.

In another aspect, the invention also relates to the use of a multimeric compound as defined above, for the in vitro or ex vivo imaging.

In another aspect, the invention relates to a method for the diagnosis of a pathology, comprising a step of administering a multimeric compound as defined above, to a patient, said pathology being chosen among: cancers, diseases of epithelial organs including acute and chronic liver diseases, acute and chronic kidney diseases, chronic lung diseases and chronic skin wounds, diseases of the central nervous system including neuron diseases and sclerosis, ischemic heart diseases, peripheral vascular diseases, diabetes and associated complications such as peripheral neuropathies.

In diagnostic methods and medical imagery, multimeric compound can be detected and quantified in biological samples by dosage (for example using a biopsy) or by pictures (obtained from technologies such as PET scan or IRM).

In another aspect, the invention relates a method for medical imaging comprising a step of administering a multimeric compound as defined above, to a patient.

In one embodiment, the invention also relates to a method for medical imaging, wherein said multimeric compound allows the detection of the tyrosine kinase receptor MET.

In one embodiment, the invention also relates to a method for medical imaging, wherein said multimeric compound allows the pretargeting of an antibody.

Indeed, the multimeric compound of the invention can be linked to an antibody that recognizes a specific epitope of a tracer.

In another embodiment, the invention also relates to a method for medical imaging, wherein said multimeric compound allows the detection of a biotinylated tracer.

Such a capacity of the multimeric compound results from the capacity of the streptavidin derivatives to bind biotin and therefore, biotinylated molecules.

In another aspect, the invention relates to a pharmaceutical composition comprising a multimeric compound as defined above, in association with a pharmaceutically acceptable vehicle.

In another aspect, the invention relates to a multimeric compound as defined above, for use as a medicament.

In another aspect, the invention relates to a multimeric compound as defined above, for use in the treatment of tissue injuries by promoting cell survival or tissue regeneration.

In another aspect, the invention relates to a multimeric compound as defined above, for use in the treatment of a pathology chosen among: diseases of epithelial organs including acute and chronic liver diseases, acute and chronic kidney diseases, chronic lung diseases and chronic skin wounds, diseases of the central nervous system including neuron diseases and sclerosis, ischemic heart diseases, peripheral vascular diseases, diabetes and associated complications such as peripheral neuropathies.

In one embodiment, the invention relates to a multimeric compound, for use in the treatment of tissue injuries, or for use in the treatment of a pathology as defined above, said multimeric compound being administrable at a dose comprised from about 1 mg/kg to 1 g/kg, preferably from about 10 mg/kg to about 100 mg/kg.

In one embodiment, the invention relates to a multimeric compound, for use in the treatment of tissue injuries, or for use in the treatment of a pathology as defined above, said multimeric compound being used under a form liable to be administrable by oral or intraveinous route at an unitary dose comprised from 1 mg to 1,000 mg, in particular from 10 mg to 1,000 mg, in particular from 100 to 1,000 mg.

In particular, the multimeric compound can be administrable at an unitary dose of 1 mg, 5 mg, 10 mg, 20 mg, 30 mg, 40 mg, 50 mg, 100 mg, 150 mg, 200 mg, 250 mg, 300 mg, 350 mg, 400 mg, 450 mg, 500 mg, 450 500 mg, 550 mg, 600 mg, 650 mg, 700 mg, 750 mg, 800 mg, 850 mg, 900 mg, 950 mg or 1000 mg.

In another aspect, the invention also relates to the use of a multimeric compound to promote angiogenesis, in in vivo, ex vivo or in vitro conditions.

In another aspect, the invention relates to the use of a multimeric compound as defined above, as an in vitro research tool.

In another aspect, the invention relates to a molecular complex between a multimeric compound as defined above and a tyrosine kinase receptor MET, said multimeric compound being complexed with said tyrosine kinase receptor MET by at least two K1 domains.

The invention will be better explained by the following figures and examples. In any case, the following examples should not be considered as restricting the scope of the invention.

### LEGENDS TO THE FIGURES

FIG. 1. K1B total chemical synthesis. (a) Structure of the K1 domain of HGF/SF (residues 125-209, extracted from PDB 1BHT). The annotation was done according to UniProt database (entry P14210) with the 3 internal cysteine bridges and C-term biotin. (b) Scheme of one-pot assembly and folding of K1B. (c) RP-HPLC characterization of the crude linear K1B domain (left), the purified K1B domain (center) and MS analysis of folded K1B domain (right).

FIG. 2. HeLa cells were treated for 7 min with 100 pM or 500 pM HGF/SF (HGF), 100 nM or 1 μM K1 and 100 nM or 1 μM K1B. Cell lysates were then analyzed by specific total MET, Akt and ERK or phospho-MET, phospho-Akt and phospho-ERK western blot.

FIG. 3. Cell scattering assay. MDCK isolated cell islets were incubated for 18 h in presence of culture media (Ctrl) with 1 μM K1 or 1 μM K1B. Cells were then stained and observed under microscope (40×).

FIG. 4. K1B and NB MET binding properties. (a) Structure of NK1 dimer (center, PDB 1BHT) and spatial relative orientation of each N (left) and K1 (right) monomers within the dimer. Dashed arrows indicate distances between subdomain C-termini. (b) NB, K1B and MET-Fc binding assay. Increasing concentrations of NB or K1B were mixed with extracellular MET domain fused with human IgG1-Fc (MET-Fc), and incubated with streptavidin AlphaScreen donor beads and Protein A acceptor beads. Error bars correspond to standard error (+/−SD) of triplicates. (c) Endogenous MET capture. Streptavidin coated beads loaded with NB or K1B were incubated with HeLa or Capan-1 total cell lysates. Input, flow-through and elution fractions from NB or K1 loaded beads were analyzed by specific total MET western blot.

FIG. 5. Structure of a streptavidin homotetramer with 4 bound biotins (left, PDB 1SWE) and distances between binding sites (right).

FIG. 6. AlphaScreen competition assay. Increasing concentrations of K1B/S complex (ratio 2:1) were added to pre-mixed K1B (20 nM)/MET-Fc (2 nM)/Alpha beads. IC50 of Alpha signal was measured. Graph is representative of experiments reproduced at least 3 times with 2 different lots of K1B. Error bars correspond to standard error (+/−SD) of triplicates.

FIG. 7. Analysis of K1B/S complexes. Increasing ratio of K1B and streptavidin (from 0:1 to 8:1) were analyzed in non-denaturing condition by SDS-PAGE on a 10% NuPage® gel in MES buffer. Gel was fixed and stained with Coomassie Brilliant Blue. K1B:S ratio for each complex composition is indicated with corresponding A, B, C and D relative biotin binding sites positioned as proposed in FIG. 6.

FIG. 8. (a) Mass spectrum of K1B under native conditions. (b) Titration of streptavidin with K1B. Upon addition of K1B, new species corresponding to the binding of 1 to 4 molecules of K1B to the streptavidin are clearly visible. (c) Relative intensity of each species depending on the K1B:S ratio.

FIG. 9. Determination of optimal K1B:S ratio. HeLa cells were treated for 7 min with 50 nM streptavidin (S), 500 pM mature HGF/SF (HGF), 400 nM K1B and an increasing ratio of K1B/S mixture (from 1:1 to 8:1) with 50 nM streptavidin. Cell lysates were then analyzed by specific total MET, Akt and ERK or phospho-MET, phospho-Akt and phospho-ERK western blot.

FIG. 10. Structure of human IgG: distance between two paratopes is 13.7 nm (PDB 1IGt).

FIG. 11. MET signaling analysis upon K1B/S stimulation. (a) HeLa cells were treated for 7 min with 50 nM streptavidin (S), 50 nM anti-biotin antibody (Ab), 500 pM mature HGF/SF (HGF), 100 nM and 1 μM K1B, 100 nM K1B/S, 100 nM K1B/Ab and 100 nM NK1. Cell lysates were then analyzed by specific total MET, Akt and ERK or phospho-MET, phospho-Akt and phospho-ERK western blot. Ctrl: vehicle, MW: molecular weight. (b) HeLa cells were treated with increasing concentrations of mature HGF/SF, K1B/S, NK1 and K1B/Ab for 7 min. Activation levels of ERK and Akt were measured using HTRF technology, and plotted as the 665/620 nm HTRF signal ratio. (c) K1B/S and NK1, K1B/Ab kinetic analysis. HeLa cells were treated with 100 nM K1B/S or NK1, for 1, 5, 10, 20, 30, 40 or 90 min. Cell lysates were then analyzed by specific total MET, Akt and ERK or phospho-MET, phospho-Akt and phospho-ERK western blot. (d) HGF/SF, K1B/S, NK1 and K1B/Ab kinetic analysis. HeLa cells were treated with optimal concentration of 100 pM HGF/SF, 50 nM K1B/S, 50 nM NK1 or 400 nM K1B/Ab for 1, 3, 5, 7, 10, 15, 20, 30, 60 or 90 min. Activation levels of ERK and Akt were measured using HTRF technology and plotted as the 665/620 nm HTRF signal ratio.

FIG. 12. Analysis of MET tyrosine phosphorylation profile. HeLa cells were treated for 7 min with 50 nM streptavidin (S), 50 nM anti-biotin antibody (Ab), 500 pM mature HGF/SF (HGF), 10 or 100 nM K1B, 100 nM K1B/S, 100 nM K1B/Ab or 100 nM NK1. Cell lysates were then analyzed by western blot with total MET and phospho-specific MET Y1234-1235 and Y1349-1356 residues.

FIG. 13. HGF/SF, K1B/Ab kinetic analysis. HeLa cells were treated with 500 pM HGF/SF or 100 nM K1B/Ab, for 1, 5, 10, 20, 30, 40 or 90 min. Cell lysates were then analyzed by specific total MET, Akt and ERK or phospho-MET, phospho-Akt and phospho-ERK western blot.

FIG. 14. HeLa cells were treated for 7 min with 100 pM HGF/SF (HGF), 1 μM NB and 1 μM NB/S (2:1 ratio), and 500 nM Streptavidin (S). Ctrl: vehicle. Cell lysates were then analyzed by specific total MET, Akt and ERK or phospho-MET, phospho-Akt and phospho-ERK western blot.

FIG. 15. Cell scattering assay. MDCK isolated cell islets were incubated for 18 h in culture media (Ctrl), 500 pM HGF/SF (HGF) 500 nM streptavidin (S), 1 μM NB or 1 μM NB/S. Cells were then stained and observed under microscope (40×).

FIG. 16. Cellular phenotypes induced by K1B/S. (a) Cell scattering assay. MDCK isolated cell islets were incubated for 18 h in culture media with 50 nM streptavidin (S), 50 nM anti-biotin antibody (Ab), 500 pM mature HGF/SF (HGF), 100 nM K1B, 100 nM K1B/S, 100 nM NK1 and 100 nM K1B/Ab. Cells were then stained and observed under microscope (40×). (b) Matrigel morphogenesis assay. MDCK cells were seeded onto a layer of Matrigel and treated for 18 h with 50 nM streptavidin (S), 50 nM antibiotin antibody (Ab), 500 pM mature HGF/SF (HGF), 100 nM K1B, 100 nM K1B/S, 100 nM NK1 and 100 nM K1B/Ab. Cells were then observed under microscope (40×). (c) MTT Assay. MDCK cells were cultured overnight (15 h) in medium containing 0.1% FBS with or without anisomycin (0.7 μM) and in the presence of 500 pM mature HGF/SF (HGF), 100 nM K1B, 100 nM K1B/S, 100 nM NK1 and 100 nM K1B/Ab. An MTT assay was then performed to evaluate cell survival. Results are expressed as the percentage of untreated control. An ANOVA test was performed to compare the 3 means, with a P-value <0.05 considered statistically significant. (d) Angiogenesis. Mice were injected with a mixture of Matrigel and 1 nM HGF/SF (HGF), 10 nM VEGF, 100 nM NK1, 100 nM K1B/S, 100 nM K1B or 50 nM S. Hemoglobin absorbance was measured and concentration was determined using a rate hemoglobin standard curve and plug weight. ANOVA tests were performed to compare all the means, and a P-value<0.001 was considered to indicate a statistically significant difference.

FIG. 17. In vivo MET activation assays. (a) FVB mice were injected intravenously with PBS (ctrl), 25 pmol K1B (250 ng), 25 pmol K1B/S complex (250 ng K1/700 ng S), 25 pmol NK1 (500 ng) or 2.5 pmol mature HGF/SF (250 ng) per g of body weight. After 10 min, livers were extracted, snap frozen and crushed. MET, Akt and ERK phosphorylation status in cell lysates was analyzed by western blot. Data obtained from 2 mice are representative of 3 independent experiments. (b) FVB mice were injected intravenously with 125 ng anti-Fas monoclonal antibody (aFas) mixed with 25 pmol K1B (250 ng), 25 pmol K1B/S complex (250 ng/700 ng), 25 pmol NK1 (500 ng) or 2.5 pmol mature HGF/SF (250 ng) per g of bodyweight, or PBS. A second injection without anti-Fas was performed 90 min later.

Livers were extracted and fixed in formalin after 3 additional hours. (c) Frozen liver sections were stained with hematoxylin-eosin for histological observation (40×). (d) Frozen liver sections were treated with Apoptag® Kit for apoptotic nuclei labelling (green) and counterstained with DAPI for total nuclei labelling (blue) (100×, insert: 200× on apoptotic cells).

FIG. 18. Mice were injected with an increased concentration of K1B/S complex (0.5, 2.5 or 25 pmol/g, corresponding to 5 ng K1B/14 ng S, 25 ng K1B/70 ng S and 250 ng K1B/700 ng S), 25 pmol K1B/g (250 ng/g) or 25 pmol/g NK1 (500 ng/g). After 10 min, livers were extracted, snap frozen and crushed. Cell lysates were analyzed by specific total MET, Akt and ERK or phospho-MET, phospho-Akt and phospho-ERK western blot.

FIG. 19. In vivo MET activation kinetics. Mice were injected with 25 pmol K1B/S (250 ng/700 ng) per g of body weight, and livers were extracted after 0, 10, 20 or 30 min, snap frozen and crushed. Cell lysates were analyzed by specific total MET, Akt and ERK or phospho-MET, phospho-Akt and phospho-ERK western blot.

FIG. 20. Fas-induced fulminant hepatitis. FVB mice were injected intravenously with 125 ng anti-Fas monoclonal antibody (aFas) mixed with 25 pmol K1B, 25 pmol K1B/S complex, 25 pmol NK1, 12.5 pmol Streptavidin (S) or 2.5 pmol mature HGF/SF per g of body weight, or PBS. A second injection without anti-Fas was performed 90 min later. Livers were extracted, snap frozen and crushed. Proteins were analyzed by specific total MET, PARP 1/2, Caspase 3, cleaved Caspase 3 and total ERK western blot.

### EXAMPLES

### Example 1. Total Chemical Synthesis of Biotinylated K1 and N Domains

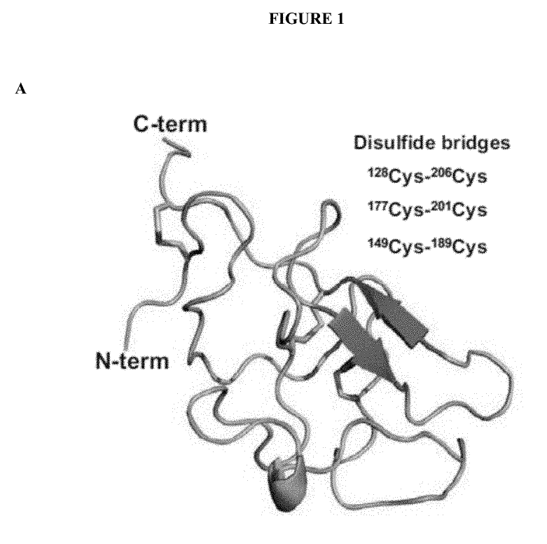

The K1 domain (HGF/SF 125-209) is composed of 85 amino acid residues, and its tertiary structure is stabilized by three disulfide bonds (FIG. 1A). In K1B, the K1 primary structure was extended at the C-terminus by addition of two glycine residues and a lysine residue modified on its side chain with a biotin group. The chemical synthesis of K1B was performed using solid phase peptide synthesis (SPPS) in a one-pot sequential three peptide segments assembly process, which required the preparation of HGF/SF segments 125-148 (segment 1), 149-176 (segment 2) and 177-209 (segment 3), the latter with the GGK (biotin) extension (FIG. 1B). A thioester and bis(2-sulfanylethyl)amido cyclic disulfide (SEAoff) group were introduced on the C-terminus of peptide segments 1 and 2 respectively. Assembly of K1B linear polypeptide started by joining thioester segment 1 with segment 2 using the Native Chemical Ligation reaction. The reaction led to the successful formation of segment 1-2 featuring a blocked C-terminal SEAoff group. Activation of the SEAoff group by reduction with tris(2-carboxyethyl)phosphine (TCEP) and addition of biotinylated segment 3 triggered the SEA native peptide ligation step and the successful formation of linear K1B HGF/SF domain as shown by the LC-MS of the crude reaction mixture (FIG. 1C, left). Linear K1B was purified by HPLC to give 3.6 mg (40% overall) of homogeneous material (FIG. 1C, center) and folded subsequently using the glutathione-glutathione disulfide redox system. Proteomic analysis of the folded K1B domain demonstrated the formation of the native disulfide bond pattern (FIG. 1C, right).

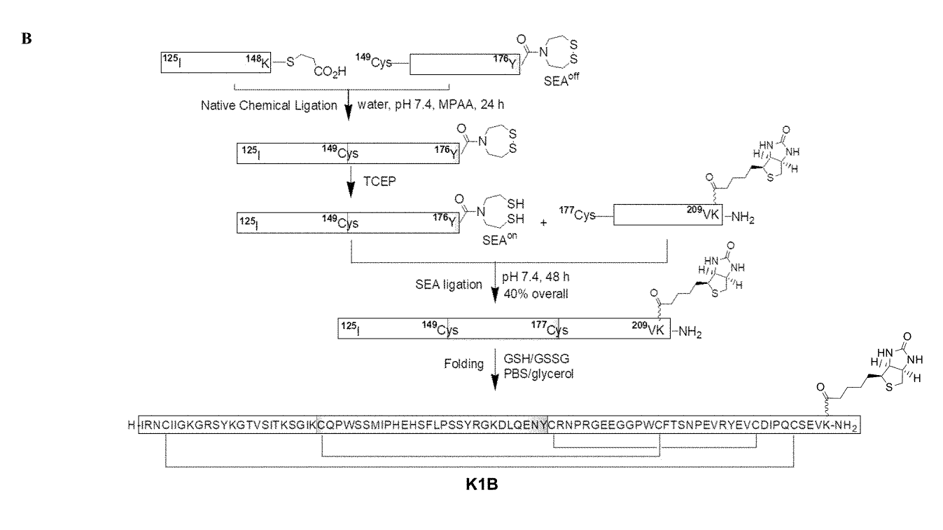

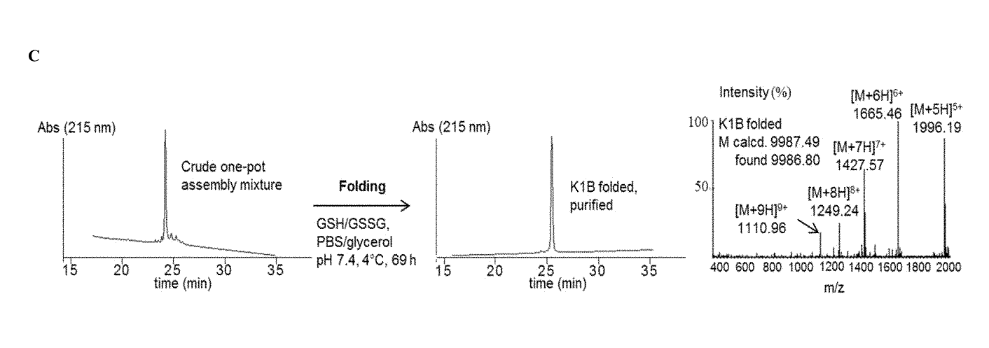

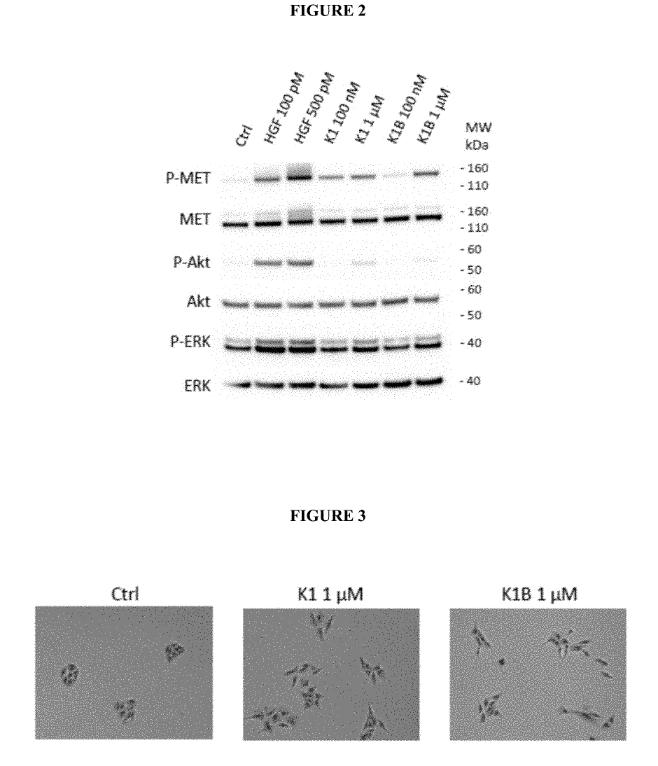

Interestingly, a MET phosphorylation assay using HeLa cells (FIG. 2) and cell scattering assays using MDCK cells (FIG. 3) showed that K1B activity was indistinguishable from unmodified synthetic K1 domain and behaved as a micromolar MET agonist, as it is known for recombinant K1 domain. Consequently, introduction of the biotin group had no detectable influence on the biological activity of K1B at this stage.

### Example 2. Design of K1 Multivalent Complexes

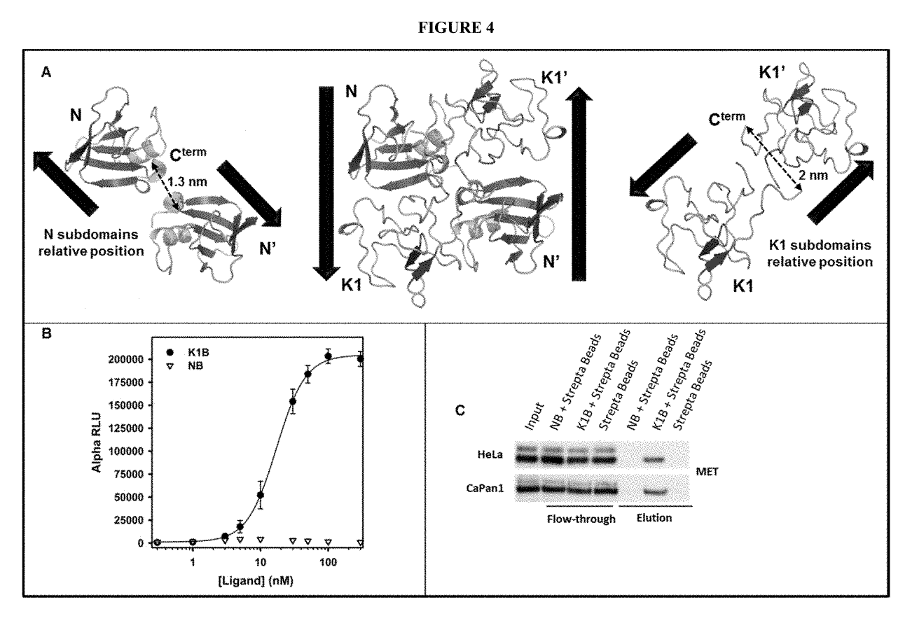

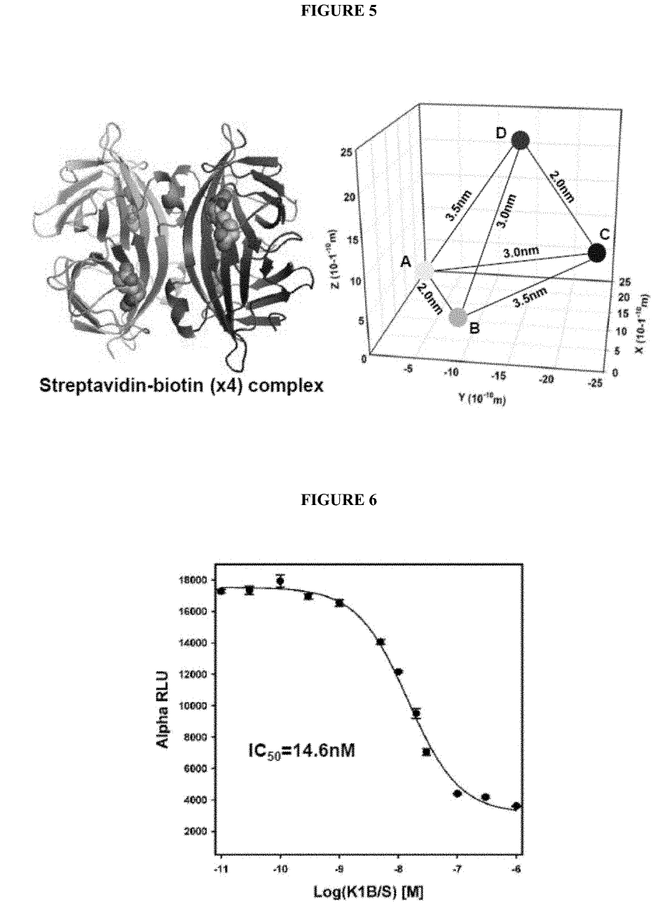

Analysis of the relative positions of N and K1 domains in the NK1 homodimer crystal structure reveals that the C-termini of the two N domains and the C-termini of the two K1 domains are separated by only ˜1.3-2 nm (FIG. 4A). Interestingly, the individual biotin binding sites within a streptavidin homotetramer (S) are separated by distances of ˜2.0-3.5 nm (FIG. 5). Therefore, it was anticipated that the formation of K1B/S or NB/S complexes might recapitulate the relative distances and positions of N and K1 domains found in NK1 dimer independently of each other.

The binding of K1B/S complexes to MET was examined using AlphaScreen technology. K1B was loaded on streptavidin-coated donor beads and incubated with recombinant extracellular MET-Fc chimera loaded on Protein A-coated acceptor beads. If K1B/S donor beads interact with MET-Fc/Protein A acceptor beads, a chemical energy transfer is possible between the beads, leading to fluorescence emission upon laser excitation. K1B induced strong signal intensities with an apparent dissociation constant KD (˜16 nM) about 100-fold lower than the KD reported for monomeric K1 protein-MET interaction (FIG. 4B). Since the bead-based AlphaScreen assay can generate avidity and thus introduce a bias in the estimation of the apparent KD in saturation experiments, it was performed the reciprocal competition assay by adding increasing concentrations of preformed K1B/S complex (2:1 molar ratio) into the K1B/MET-Fc/AlphaScreen bead mixture (FIG. 6). With this competition assay, an IC50 (˜14 nM) was determined in perfect agreement with the apparent K1B/MET-Fc KD from the saturation assay. This study was completed by examining the binding of K1B/S complexes to endogenous MET from a whole cell lysate (FIG. 4C). Streptavidin-coated agarose beads were incubated with K1B to form immobilized complexes, which were subsequently incubated with whole lysate from HeLa or Capan-1 cells. Western blot analysis of the eluted material showed that K1B/S complexes were able to capture MET from cell lysates. Collectively, these data show that the semisynthetic K1B/S complex interacts with MET at low nanomolar concentration, and indicate the importance of multivalency in the K1-MET interaction system.

### Example 3. Semisynthetic K1B/S Complex is a Potent MET Agonist

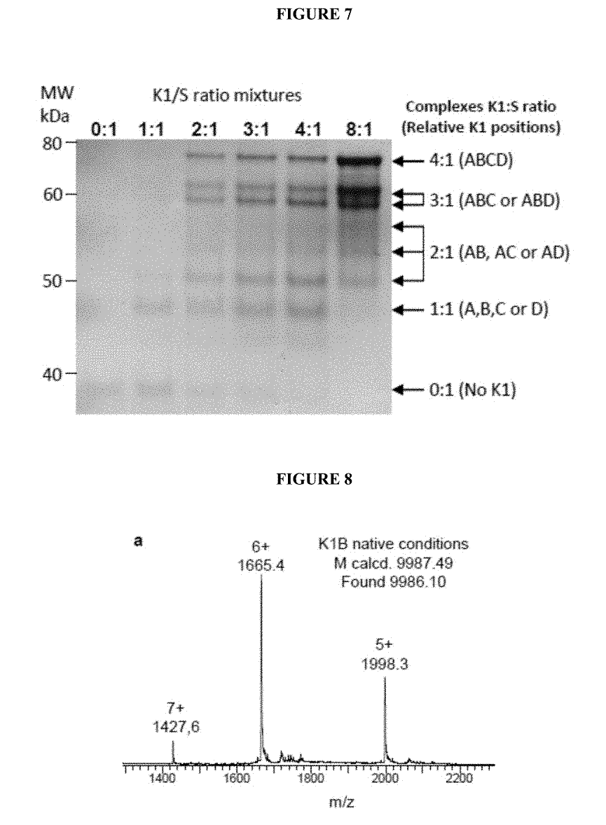

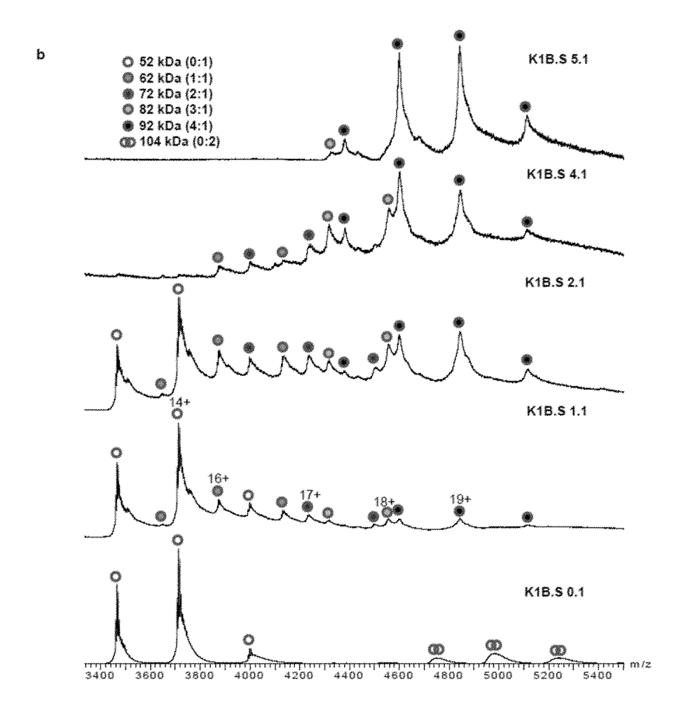

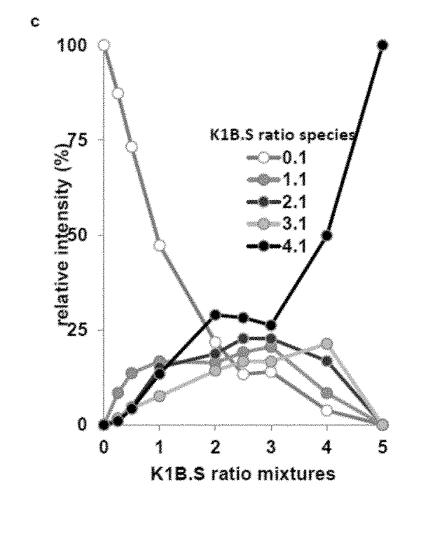

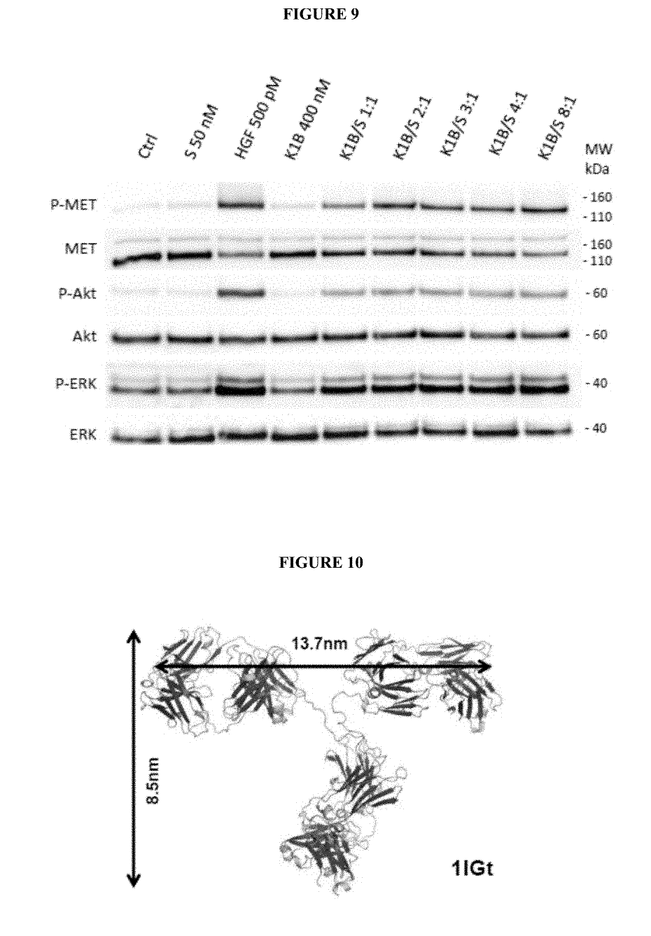

These results set the stage for evaluating the K1B/S complex agonistic activity using in vitro cell assays in the human HeLa cell line. For this, the stoichiometry for K1B/S complex formation was fixed to 2:1, which generates several species varying in the number of K1B proteins bound per streptavidin tetramer. With this molar ratio, and by assuming that each biotin binding unit is independent, the probability of having 0, 1, 2, 3 or 4 K1B proteins bound per streptavidin should correspond to 6%, 25%, 38%, 25% and 6% respectively, meaning 69% of K1B/S multimers in theory. These K1B/S multimers were indeed identified by SDS-PAGE analysis (FIG. 7) and by native mass spectrometry analysis (FIG. 8). Using the latter technique, it was estimated that the 2:1 K1B:S molar ratio resulted in 75% of the K1 domain presented at least as pairs within K1B/S multimers. In practice, it was noticed that a 2:1 K1B:S molar ratio was sufficient to achieve a maximum cellular response, since a higher proportion of K1B in the mixture from 3:1 up to 8:1 led to no improvement in potency (FIG. 9).

Another complex produced by mixing K1B with an anti-biotin antibody (Ab) in a 2:1 molar ratio was also designed. The antibody is expected to produce consistent K1B dimers, albeit with a distance of ˜13-20 nm between each K1B protein, which is significantly greater than those found in NK1 crystal structure or K1B/S complexes (FIG. 10).

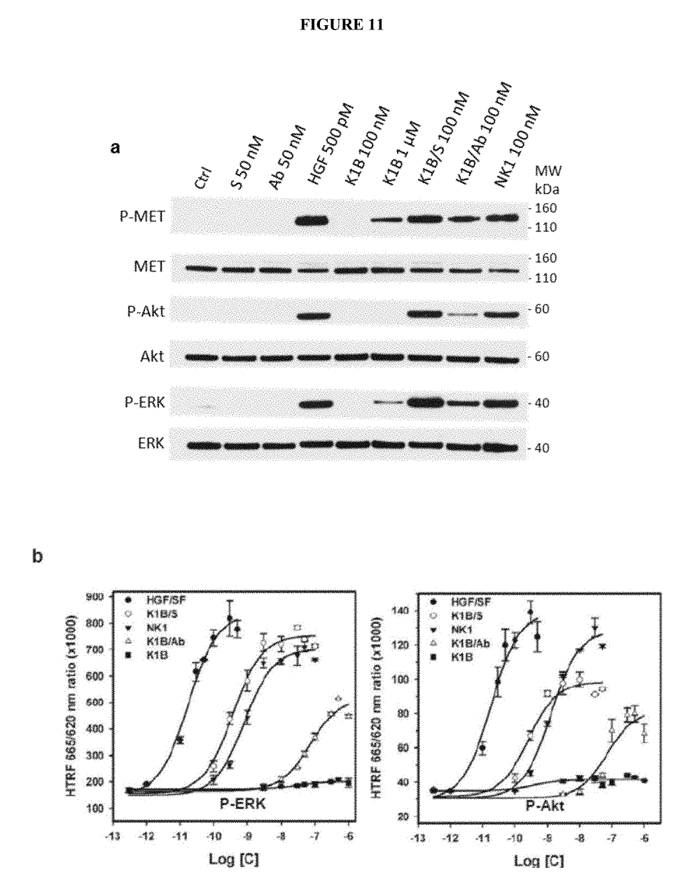

MET activation and downstream signaling in HeLa cells upon HGF/SF, K1B, K1B/S, K1B/Ab or recombinant NK1 incubation was analyzed by western blot and quantified by HTRF approaches (FIGS. 11A & B). Typically, HGF/SF triggered maximal ERK and Akt activation down to pM concentrations. Impressively, K1B/S complexes were able to trigger ERK and Akt phosphorylation levels down to a low nM range, and thus displayed an agonist activity similar to NK1 protein. Moreover, K1B/S but not K1B induced a strong MET phosphorylation at 100 nM. The fact that activation of MET by K1B was detected only for μM concentrations, as reported in the literature for recombinant K1, highlights the critical role of multivalency for achieving strong receptor activation. A similar multivalent process was evident for the K1B/Ab complex, which unlike K1B, also induced a significant MET phosphorylation at 100 nM.

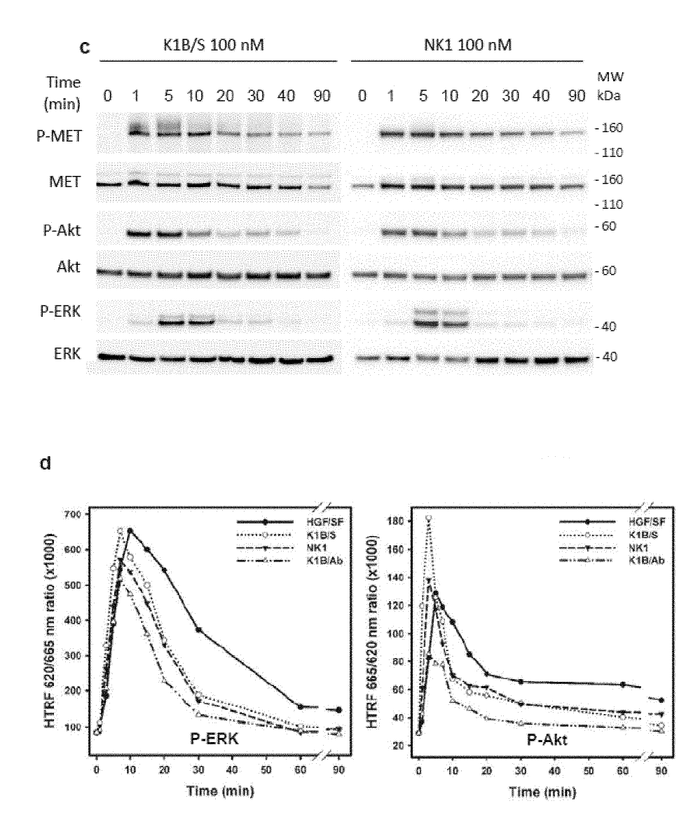

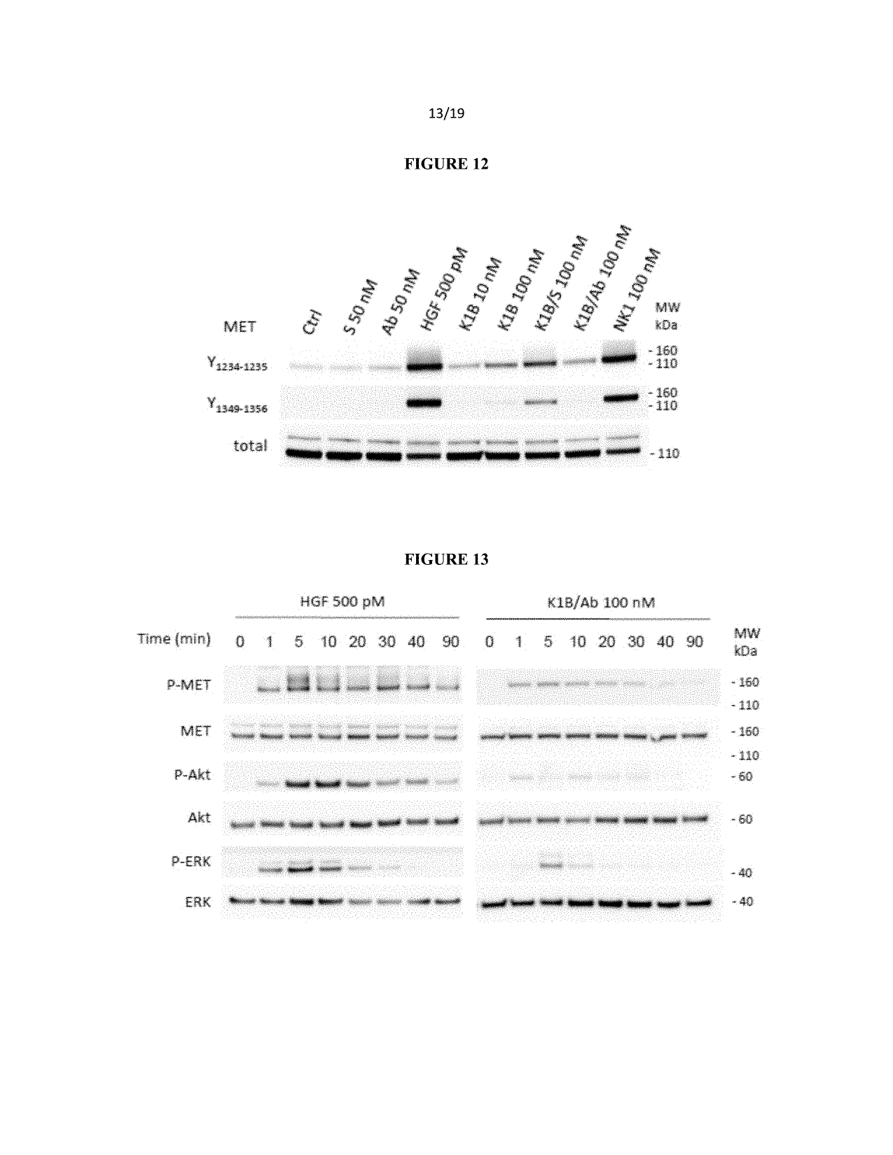

However, K1B/Ab was significantly less active than K1B/S since it was unable to trigger significant ERK and Akt downstream signaling (FIG. 11A). The MET phosphorylation pattern was analyzed at the tyrosine level. Indeed, auto-phosphorylation of tyrosines 1234 and 1235 is the first event leading MET activation, and is crucial for unlocking and maintaining sustained kinase activity. Subsequently, phosphorylation of C-terminal tyrosines 1349 and 1356 is required to provide recognition sites for scaffolding partners that propagate, amplify and diversify MET signaling. Both K1B/Ab and K1B/S activated MET auto-phosphorylation onto tyrosines 1234 and 1235. However, unlike K1B/S, K1B/Ab failed to trigger phosphorylation of tyrosines 1349 and 1356 (FIG. 12), and thus failed to trigger the downstream signaling cascade. This fact might be due to the large distance between K1B domains in the antibody complex and thus to the suboptimal stabilization of MET dimers.

It was also determined the MET and downstream signaling activation kinetics (0-90 min) using western Blot (FIG. 11C) and HTRF (FIG. 11D). Typically, HGF/SF induced a maximum of MET autophosphorylation between 5 and 10 min (FIG. 13), followed by a maximum of Akt and ERK phosphorylation at around 10-15 min, which slowly decreased over time. In comparison, MET phosphorylation proceeded much faster with K1B/S and NK1, i.e. within the very first minute, and then decreased below HGF/SF levels. Accordingly, maximum ERK and Akt activation was observed earlier, after only 3-7 min. In contrast, K1B/Ab complex induced weak MET activation (FIG. 13), and downstream signaling faded faster than for HGF/SF, NK1 or K1B/S.

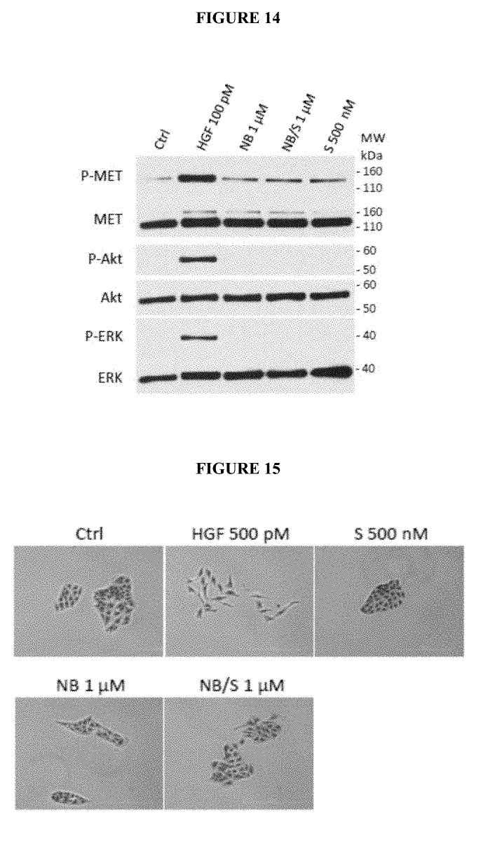

Finally, and as expected from binding experiments, NB/S complex showed no agonistic activity (FIG. 14), and did not promote any cellular phenotypes (FIG. 15).

Together these results indicate that K1B/S complex recapitulates NK1 agonist activity, and demonstrate that K1 is the minimal HGF/SF functional domain required for MET activation. Moreover, these data show that the distance and/or orientation which separates the two K1 domains within a dimeric structure (natural or synthetic) is important to induce full MET activation.

### Example 4. K1B/S Promotes Cell Scattering, Morphogenesis, Survival and Angiogenic Phenotypes

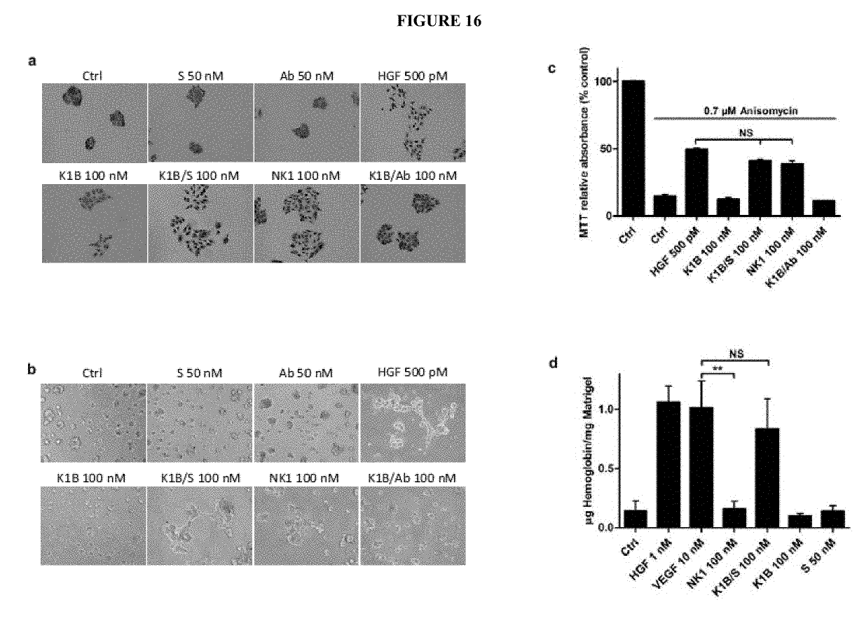

The ability of MET agonists to induce cell scattering in MDCK cells (the reference cell line for this phenotypic assay) was evaluated (FIG. 16A). In the presence of HGF/SF (100 pM) for 18-24h, MDCK cells acquired a mesenchymal-like phenotype and scatter.

This marked phenotype was also induced by NK1 protein and K1B/S complex, whereas scattering with K1B and K1B/Ab was weak. Notably, the ability of the agonists to induce a scattering phenotype seemed to be strongly correlated with their capacity to induce sustained phosphorylation of MET, ERK and Akt kinases.

Further cell assays were performed using lumina basal like matrix (Matrigel) as a mimic of basement extracellular matrix. In these conditions and without treatment, MDCK cells spontaneously form tight spherical clusters on Matrigel within 24 h. In contrast, when stimulated with HGF/SF, MDCK cells self-organize into branched and connected structures. Notably, NK1 and K1B/S widely promoted the formation of such structures (FIG. 16B), while K1B and K1B/Ab were unable to do so.

The capacity of the agonists to promote the survival of cells after apoptotic stress was examined. This phenotype is a hallmark of HGF/SF, which can protect many cell types against death induced by serum depletion, ultra-violet radiation, ischemia or some chemical substances. MDCK cells were stressed using anisomycin, a DNA and protein synthesis inhibitor which induces apoptosis. Anisomycin treatment induced ˜90% of cell death after 16 h, but only 50% of cell death when pretreated with HGF/SF (FIG. 16C). K1B/S or NK1 displayed similar survival rates, whereas K1B or K1B/Ab complex failed to protect the cells to a significant extent.

Clearly, these results show that in vitro K1B/S fully mimics the properties of NK1 as a potent MET agonist. To extend this observation in vivo, the different agonists were injected subcutaneously with Matrigel plugs into immunodeficient SCID mice to induce angiogenesis. Indeed, HGF/SF is a potent angiogenic factor that stimulates endothelial cell proliferation and migration. The plugs were extracted after 11 days to determine the quantity of hemoglobin infiltrated into the plug as a measure of angiogenesis induced (FIG. 16D). As expected, VEGF or HGF/SF showed potent angiogenic properties compared to control plugs. K1B/S induced the formation of vessels with a hemoglobin content comparable to that of VEGF and significantly higher than those induced by NK1 or K1B. Thus, while NK1 and K1B/S displayed similar potencies in in vitro cell assays, their angiogenic properties were significantly different in vivo.

### Example 5. The K1B/S Complex Activates MET in the Liver and Impairs FAS-Induced Fulminant Hepatitis

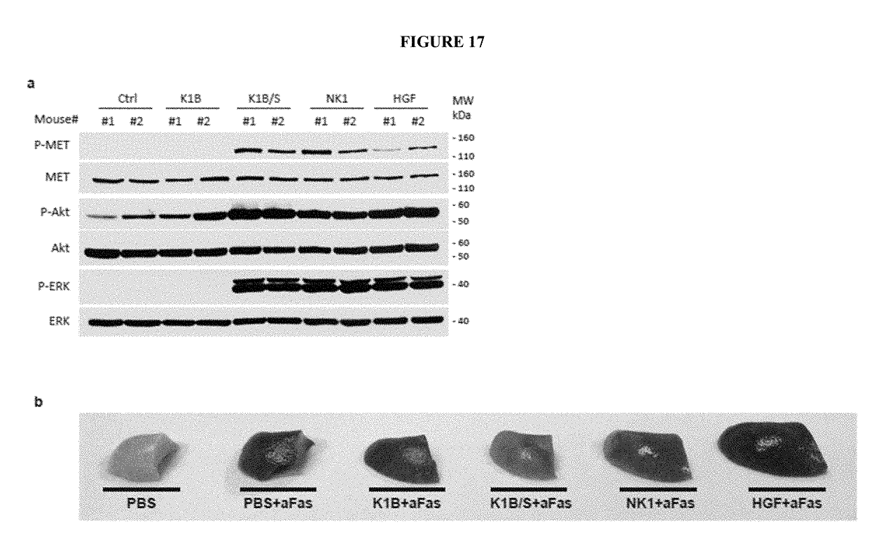

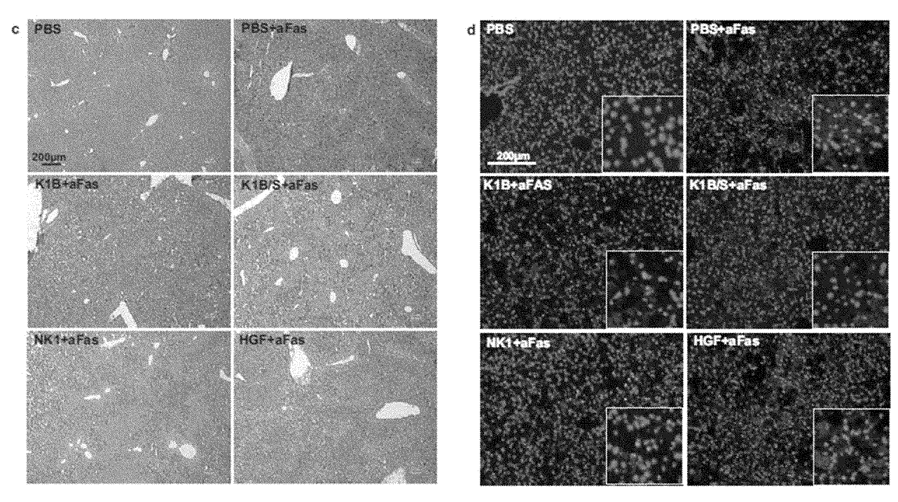

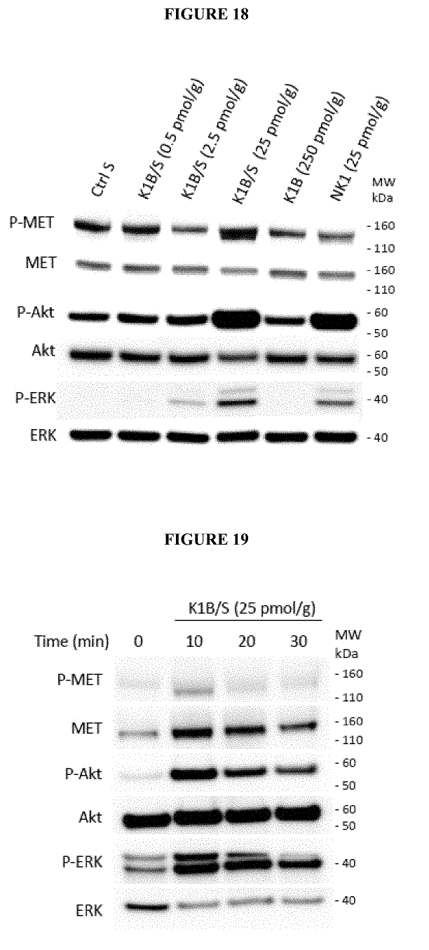

In this last assay it was examined whether the K1B/S complex could act in vivo on distant tissues when injected systemically, and thus could constitute a basis for designing potent MET agonists of potential therapeutic interest. In a first approach, the different agonists were injected intravenously to see if they could activate MET and downstream pathways in the liver, an organ well known to strongly express MET receptor. After 10 min, livers were extracted and MET, ERK and Akt phosphorylation status was determined by western Blot (FIG. 17A). K1B/S, NK1 and HGF/SF injection induced a clear MET phosphorylation associated with a strong Akt and ERK activation in the liver. Importantly, activation by K1B/S was detectable at doses as low as 2.5 pmol (250 ng) per mg of body weight (FIG. 18) and even up to 30 min post-injection (FIG. 19). In contrast, K1B and streptavidin control led to no detectable signal.

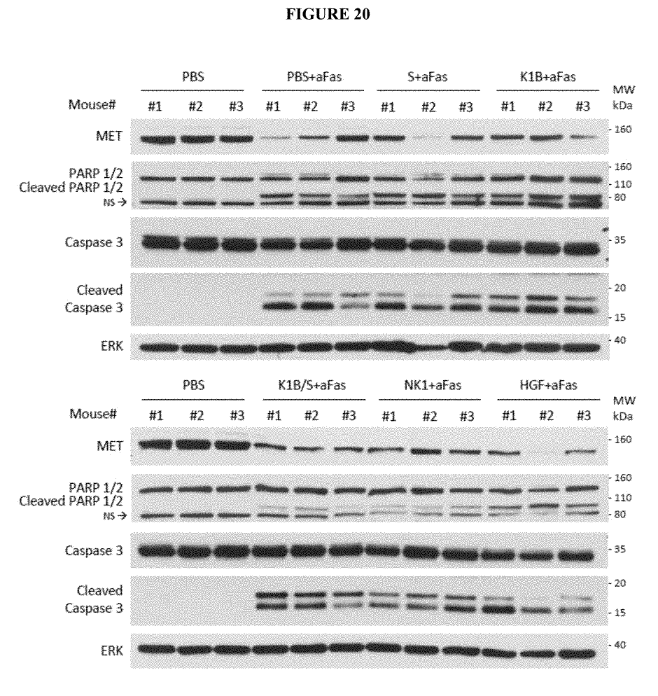

Considering the fact that K1B/S complex is able to diffuse into the liver through the blood circulation and induce MET activation, it was examined whether the complex could promote hepatocyte survival when an apoptotic stress was induced in the liver. Indeed, injection of an anti-FAS antibody (anti-CD95) in mice quickly induces a massive hepatocellular apoptosis leading to fulminant hepatitis and death of the animals. Previous studies showed that HGF/SF was able to abrogate FAS induced fulminant hepatitis, but required prohibitive amounts to show significant effects (usually 1 nmol, i.e. ˜100 μg per mouse). In the present assay, anti-FAS antibody was mixed with 25 pmol of K1B, K1B/S or NK1, or 2.5 pmol of mature HGF/SF per mg of body weight. These concentrations were sufficient to promote strong MET signaling for at least 30 min. After 90 min, a second injection of each protein was performed to sustain signaling. Livers were extracted after 3 additional hours for histological and molecular analysis. Macroscopically, mice treated with anti-FAS antibody and K1B, NK1 or mature HGF/SF presented an altered liver, retaining a deep brown color even after PBS perfusion and elimination of vascular blood content (FIG. 17B). Remarkably, mice treated with K1B/S maintained a clear liver, almost intact. Histological analysis demonstrated that this dark color was mostly induced by a vascular congestion attributable to a massive hepatocyte loss and subsequent blood infiltration. Controls and HGF/SF treated mice showed totally disorganized livers with significant blood infiltration. In contrast, K1B/S mice kept well organized structures, although some blood infiltration could be visualized. NK1 treated mice presented an intermediate phenotype, retaining some organized areas but with massive blood infiltration. Further analysis confirmed that these disorganized regions corresponded to large clusters of apoptotic hepatocytes (FIG. 17D). Interestingly, all the mice challenged with anti-Fas antibody showed the early molecular markers characteristic for apoptosis such as cleaved caspase 3 and PARP1/2, even for the animals which were protected by K1B/S complex (FIG. 20). These results show that K1B/S does not act on the initial steps following FAS receptor activation but rather on downstream intracellular apoptotic signaling.

These histological and molecular analyses demonstrated that K1B/S complex acts systematically, efficiently activates MET signaling in the liver and is a potent survival factor even in extreme apoptotic stress conditions. The fact that K1B/S was more potent than NK1 highlights the significance of these findings for future MET agonist design.

### METHODS

Chemical Protein Synthesis

Total chemical synthesis of K1 C-terminal biotin (K1B) was performed using 3 fragments in a one-pot protocol process, as described for the synthesis of biologically active K1 domain of HGF-SF (Ollivier et al., A one-pot three-segment ligation strategy for protein chemical synthesis. Angew Chem Int Ed 51, 209-213, 2012). Final purification of the full length synthetic 88 residues polypeptide and folding with concomitant formation of the 3 disulfide bridges gave synthetic biologically active K1B. The protein was aliquoted and stored at −80° C.

Design of K1B/S Complex NK1 (entry 1BHT) and streptavidin (entry 1SWE) structures were obtained from the PDB database. Extraction of K1 domain portion, visualization and distance measurements were performed on PyMOL v1.7 software.

Binding and Competition Assay

Competition assays for binding of K1B to recombinant MET-Fc protein were performed in 384-well microtiter plates (OptiPlate™-384, PerkinElmer, CA, USA, 50 μL of final reaction volume). Final concentrations were 0-300 nM for K1B, 2.5 nM for MET-Fc, 10 μg/mL for streptavidin coated donor beads and protein A-conjugated acceptor beads. The buffer used for preparing all protein solutions and the bead suspensions was: PBS, 5 mM HEPES pH 7.4, 0.1% BSA.

For K1B and MET-Fc binding assay, K1B (10 μL, 0-1.5 μM) was mixed with solutions of hMET-Fc (10 μL, 10 nM). The mixture was incubated for 10 min (final volume 15 μL). Protein A-conjugated acceptor beads (10 μL, 50 μg/mL) were then added to the vials. The plate was incubated at 23° C. for 30 min in a dark box. Finally, streptavidin coated donor beads (10 μL, 50 μg/mL) were added and the plate was further incubated at 23° C. for 30 min in a dark box. The emitted signal intensity was measured using standard Alpha settings on an EnSpire® Multimode Plate Reader (PerkinElmer). For the competition assay: increasing concentrations of K1B/S complex (ratio 2:1) were added to pre-mixed K1B (20 nM)/MET-Fc (2 nM)/ALPHA bead (10 μg/mL) complex.

Endogenous MET Capture

Streptavidin coated beads loaded with NB or K1B were incubated with HeLa or Capan-1 total cell lysates. Input, flow-through and elution fractions from NB or K1 loaded beads were analyzed by specific total MET western blot.

Cell Culture and Drug Treatment

Madin Darby Canine Kidney (MDCK) and Human cervical cancer HeLa cells, purchased from ATCC® (American Type Culture Collection, Rockville, Md., USA), were cultured in DMEM medium (Dulbecco's Modified Eagle's Medium, Gibco, Karlsruhe, Germany), supplemented with 10% FBS (Fetal Bovine Serum, Gibco®, Life technologies, Grand Island, N.Y., USA) and 5 mL of ZellShield™ (Minerva Biolabs GmbH, Germany). Twenty-four hours before drug treatment, the medium was exchanged with DMEM containing 0.1% FBS, and cells were then treated for different times with different compounds.

Akt and ERK Phosphorylation Assay by HTRF Method

The assay was performed according to the manufacturer's protocol mentioned in HTRF® (Cisbio bioassays, Bedford, Mass., USA). Briefly, cells were plated, stimulated with different agonists (HGF/SF, NK1, K1B/S and K1B/Ab), and then lysed in the same 96-well culture plate. Lysates (16 μL) were transferred to 384-well microplates for the detection of phosphorylated Akt (Ser473) and ERK (Thr202/Tyr204) by HTRF® reagents via a sandwich assay format using 2 different specific monoclonal antibodies: an antibody labelled with d2 (acceptor) and an antibody labelled with Eu3+-cryptate (donor). Antibodies were pre-mixed (2 μL of each antibody) and added in a single dispensing step. When the dyes are in close proximity, the excitation of the donor with a light source (laser) triggers a Fluorescence Resonance Energy Transfer (FRET) towards the acceptor, which in turn fluoresces at a specific wavelength (665 nm). Upon laser excitation, energy transfer between d2 and Eu3+-cryptate molecules occurs and fluorescence is detected at 620 and 665 nm on an EnVision® Multilabel reader (PerkinElmer). Data are presented as a 620/665 nm ratio for signal normalization.

Angiogenesis

Immunodeficient SCID mice weighing 19-21 g were used for this experiment. Mice were housed in a facility with a 12 h light/dark cycle at 22° C. and had free access to food and water. Mature HGF/SF, VEGF-A, NK1, K1B, Streptavidin and K1/S complexes were added to growth factor reduced Matrigel™ (BD Biosciences, Becton Dickinson, Belgium). Mice (n=6) were injected subcutaneously in the flank with 400 μL of Matrigel. After 11 days, mice were sacrificed, Matrigel plugs were removed and weighed, and 300 μL of water was added to induce hypotonic red blood cell lysis and hemoglobin release. Hemoglobin absorbance (405 nm) was measured, and concentration was determined against a hemoglobin standard curve and plug weight.

All experimental procedures were conducted with the approval of the Ethics Committee for Animal Experimentation of the Nord Pas de Calais Region (CEEA 75).

Fas Induced Fulminant Hepatitis

FVB mice weighing 19-21 g (Charles River) were used for this experiment. After anesthesia with isoflurane (Aerrane, Baxter, USA), mice (n=3) were given intravenous injections of 125 ng/g body weight of anti-Fas antibody (Clone Jo-2, CD95, Pharmingen, BD Biosciences) mixed with different agonists (HGF/SF, NK1, and K1/S) in PBS. The mice were injected a second time with each agonist 90 min after the first injection. The mice were sacrificed after 3 additional hours, and their livers perfused with PBS supplemented with protease and phosphatase inhibitors.

In parallel, to visualize MET activation in the liver, mice were given intravenous injections of each agonist for 10 min.

For histological analysis, liver tissue was collected, fixed overnight in 4% paraformaldehyde, and snap frozen in isopentane, submerged in liquid nitrogen, and embedded in OCT (Tissue-Tek®, VWR, PA, USA). Frozen liver sections (5 μm) were stained with hematoxylin and eosin (HE) for general morphology. TUNEL staining for apoptosis was also performed on liver sections according to the manufacturer's instructions (Apoptag® Fluorescein Direct In Situ kit, Merck Millipore, Billerica, Mass., USA). For molecular analysis, extracted liver tissue was immediately frozen in liquid nitrogen. Livers were crushed in lysis buffer supplemented with freshly added protease and phosphatase inhibitors.

### Reagents and Antibodies

Recombinant human HGF/SF was purchased from Invitrogen (Breda, Netherlands), recombinant VEGF-A from R&D Systems (Minneapolis, Minn., USA), Streptavidin (Streptomyces avidinii) from ProZyme (Hayward, Calif., USA) and Anisomycin (Streptomyces griseolus) from CalbioChem (Germany). Recombinant human NK1 protein (residues 28-209) was kindly provided by Prof. Ermanno Gherardi (University of Pavia (Italy). Antibodies directed against the kinase domain of MET were purchased from Invitrogen, anti-phospho-MET (Tyr1234/1235), anti-phospho-MET (Tyr1349), anti-total Akt, anti-phospho-Akt (Ser473), anti-phospho-ERK1/2 (Thr202/Tyr204) and anti-Caspase-3 from Cell Signaling (Massachusetts, USA), anti-ERK2 (C-14) and anti-PARP1/2 from Santa Cruz Biotechnology (Santa Cruz, Calif., USA). Anti-biotin monoclonal antibody and horseradish peroxidase (HRP)-conjugated antibodies directed against rabbit or mouse IgG were purchased from Jackson ImmunoResearch Laboratories (West Grove, Pa., USA).

Characterization of K1B/S Complex

K1B and streptavidin complex ratios were analyzed by SDSPAGE using 10% NuPage precast gels run in MES buffer (Life Technologies) without heating the samples. Gels were fixed in 20% methanol and 5% acetic acid for 30 min, and stained in Coomassie Brilliant Blue solution.

Native Mass Spectrometry

Streptavidin and K1B were first buffer exchanged in 200 mM ammonium acetate pH 7.4, using Zeba™ bench-top spin desalting columns (Thermo Scientific). Protein concentrations were determined by measuring the absorbance at 280 nm and using extinction coefficients of 16,500 and 165,000 M⁻¹ cm⁻¹ for K1B and streptavidin, respectively. Titration was performed by adding 0 to 5 molar equivalents of K1B to streptavidin. A 10 μl volume was prepared per sample, and final concentrations ranged from 1 to 20 μM. Noncovalent MS analysis was performed on a Synapt G2 HDMX (Waters, Manchester, UK) coupled to an automated chip-based nanoelectrospray device (Triversa Nanomate, Advion Biosciences, Ithaca, USA) operating in the positive ion mode.

Instrument parameters were as follows: capillary, sample cone and extraction cone voltages were set at 1.55 kV, 65 V and 5 V, respectively. The backing pressure was increased to 6 mbar to improve the transmission of high molecular weight species by collisional cooling. Calibration was performed with a 2 mg/ml cesium iodide solution and data were analyzed with MassLynx software v.4.1 (Waters, Manchester, UK).

Endogenous MET Capture

HeLa and Capan-1 cells were collected by scraping and then lysed on ice with a lysis buffer (20 mM Tris HCl, 50 mM NaCl, 5 mM EDTA and 1% Triton X-100). Lysates were clarified by centrifugation (20,000 g×15 min) and protein concentration was determined (BCA protein assay Kit, Pierce®, Thermo scientific, IL, USA). Streptavidin-Sepharose beads (GE Healthcare) were washed and equilibrated in PBS. Beads were loaded with 15 μg K1B or NB (100 μl beads in a 50:50 PBS:bead slurry) for 20 min at room temperature and immediately washed with PBS. Beads were incubated with 250 μg of protein cell lysates overnight at 4° C. under mild agitation. Beads were quickly washed with PBS and bound proteins were eluted with 200 mM glycine buffer pH 2. Elution fractions were then analyzed by western blotting.

Western Blots

Cells were collected by scraping and then lysed on ice with a lysis buffer (20 mM HEPES pH 7.4, 142 mM KCl, 5 mM MgCl2, 1 mM EDTA, 5% glycerol, 1% NP40 and 0.1% SDS) supplemented with freshly added protease and phosphatase inhibitors (#P8340 and #P5726, respectively, Sigma). Lysates were clarified by centrifugation (20,000 g×15 min) and protein concentration was determined (BCA protein assay Kit, Pierce®, Thermo scientific, IL, USA). The same protein amount of cell extracts was separated by either classical SDS-PAGE or NuPAGE (4-12% or 10% Bis-Tris precast gels) (Life technologies) and electrotransferred to polyvinylidene difluoride (PVDF) membranes (Merck Millipore). Membranes were probed with indicated primary antibodies, followed by incubation with appropriate HRP conjugated secondary antibodies. Protein-antibody complexes were visualized by chemiluminescence with the SuperSignal® West Dura Extended Duration Substrate (Thermo scientific), using a LAS-3000 imaging system (Fujifilm, Tokyo, Japan) or X-ray films (CL-Xposure™ Film, Thermo scientific).

MTT Assay

Cells were washed with PBS to eliminate dead cells and then incubated in medium containing 0.5 mg/ml 3-(4,5-dimethylthiazol-2-yl)-2,5-diphenyltetrazolium bromide (MTT, Invitrogen) for 1 h. After a washing step with PBS, the formazan crystals were solubilized and mixed thoroughly with 0.04 M HCl in isopropanol. For each condition, 60 μl of formazan solution was loaded in triplicate onto a 96-well plate. Absorbance was then measured with a microplate spectrophotometer at 550 nm and 620 nm, as test and reference wavelengths, respectively. The absorbance correlates with cell number.

Scattering Assay

Cells were seeded at low density (2,000 cells/well on a 12-well plate) to form compact colonies. After treatment, when colony dispersion was observed, the cells were fixed and colored by Hemacolor® stain (Merck, Darmstadt, Germany) according to the manufacturer's instructions. Representative images were snap-captured using a phase contrast microscope with 40× magnification (Nikon Eclipse TS100, Tokyo, Japan).

Morphogenesis Assay

Cells were seeded onto a layer of Growth Factor Reduced Matrigel™ (BD Biosciences) (100,000 cells/well of a 24-well plate), treated and observed under phase contrast microscope. Representative images were snap-captured with 40× magnification (Nikon Eclipse TS100).

Statistical Analysis

Data were obtained in triplicate from at least 3 independent experiments, and expressed either as mean values or percentages of control values+/−SD or SEM depending on the experiments performed. When indicated, differences between data groups were determined by ANOVA using Prism 5 (GraphPad Software, Inc., San Diego, Calif., USA), and considered to be statistically significant for P<0.05.

1. Multimeric compound comprising at least two K1 peptide domains (Kringle 1) of the Hepatocyte Growth Factor/Scatter Factor (HGF/SF) and being represented by the formula (I): 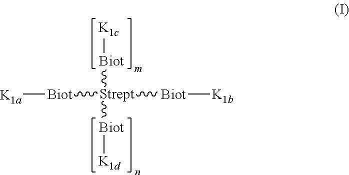
wherein:
m=0 or 1,
n=0 or 1,
K1ₐ, K1_{b}, and, if present, K1_{c} and K1_{d} are polypeptides,
K1ₐ and K1_{b} and, if present, K1_{c} and K1_{d} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule chosen among the group consisting of: streptavidin, avidin, neutravidin and any synthetic or recombinant derivatives thereof,
K1ₐ and K1_{b} and, if present, K1_{c} and K1_{d} are C-terminally linked to a Biot by a covalent bond, and each Biot is linked to Strept by a non-covalent bond,
said multimeric compound being able to induce activation of the tyrosine kinase receptor MET.

2. Multimeric compound according to claim 1, wherein Strept represents one molecule of streptavidin.

3. Multimeric compound according to claim 1, which is a K1 dimer represented by the formula (II): 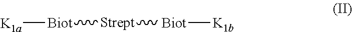
wherein:
K1ₐ and K1_{b} are polypeptides,
K1ₐ and K1_{b} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ and K1_{b} are C-terminally linked to a Biot by a covalent bond, and each Biot is linked to Strept by a non-covalent bond.

4. Multimeric compound according to claim 1, which is a K1 trimer represented by the formula (III): 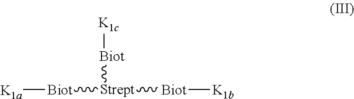
wherein:
K1ₐ, K1_{b} and K1_{c} are polypeptides,
K1ₐ, K1_{b} and K1_{c} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b} and K1_{c} are C-terminally linked to a Biot by a covalent bond, and each Biot is linked to the Strept by a non-covalent bond.

5. Multimeric compound according to claim 1, which is a K1 tetramer represented by the formula (IV): 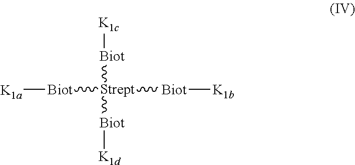
wherein:
K1ₐ, K1_{b}, K1_{c} and K1_{d} are polypeptides,
K1ₐ, K1_{b}, K1_{c} and K1_{d} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b}, K1_{c} and K1_{d} are C-terminally linked to a Biot by a covalent bond, and each Biot is linked to Strept by a non-covalent bond.

6. Multimeric compound according to claim 1, wherein K1ₐ and K1_{b}, and if present K1_{c} and K1_{d}, are identical.

7. Multimeric compound according to claim 1, wherein said multimeric compound is able to bind the tyrosine kinase receptor MET with a dissociation constant K_{D}≦200 nM, preferably ≦100 nM, more preferably ≦10 nM.

8. Composition comprising a multimeric compound as defined in claim 1.

9. Composition according to claim 8, wherein said multimeric compound is in the form of a mix of:
a K1 dimer represented by the formula (II), 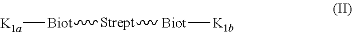
wherein:
K1ₐ and K1_{b} are polypeptides,
K1ₐ and K1_{b} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ and K1_{b} are C-terminally linked to Biot by a covalent bond, and each Biot is linked to Strept by a non-covalent bond,
a K1 trimer represented by the formula (III), 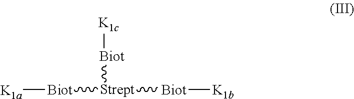
wherein:
K1ₐ, K1_{b} and K1_{c} are polypeptides,
K1ₐ, K1_{b} and K1_{c} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b} and K1_{c} are C-terminally linked to Biot by a covalent bond, and each Biot is linked to Strept by a non-covalent bond,
and, a K1 tetramer represented by the formula (IV), 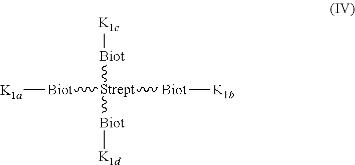
wherein:
K1ₐ, K1_{b}, K1_{c} and K1_{d} are polypeptides,
K1ₐ, K1_{b}, K1_{c} and K1_{d} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b}, K1_{c} and K1_{d} are C-terminally linked to a Biot by a covalent bond, and each Biot is linked to Biot by a non-covalent bond.

10. Multimeric compound as defined in claim 1, for use in an in vivo diagnostic method, in particular in an in vivo diagnostic method of a pathology chosen among: cancers, diseases of epithelial organs including acute and chronic liver diseases, acute and chronic kidney diseases, chronic lung diseases and chronic skin wounds, diseases of the central nervous system including neuron diseases and sclerosis, ischemic heart diseases, peripheral vascular diseases, diabetes and associated complications such as peripheral neuropathies.

11. Multimeric compound as defined in claim 1, for use in medical imaging.

12. A method for performing in vitro diagnosis, comprising providing the multimeric compound as defined in claim 1, and using the compound to perform an in vitro diagnostic of a pathology chosen among: cancers, diseases of epithelial organs including acute and chronic liver diseases, acute and chronic kidney diseases, chronic lung diseases and chronic skin wounds, diseases of the central nervous system including neuron diseases and sclerosis, ischemic heart diseases, peripheral vascular diseases, diabetes and associated complications such as peripheral neuropathies.

13. Multimeric compound as defined in claim 1, for use as a medicament.

14. Process to obtain a composition comprising a multimeric compound comprising at least two K1 peptide domains as defined in claim 1, comprising the steps of:
synthesizing a molecule containing a K1 peptide domain linked to a biotin to obtain a biotinylated K1 molecule, said biotin being linked to the C-terminus of the K1 molecule,
mixing said biotinylated K1 molecule with a streptavidin homotetramer to obtain a composition of a multimeric compound comprising at least 2 K1 peptide domains,
said biotinylated K1 molecule and said streptavidin homotetramer being preferably mixed in a 2:1 molar ratio to obtain dimeric compounds of K1 domains, a 3:1 molar ratio to obtain trimeric compounds of K1 domains, or a 4:1 molar ratio to obtain tetrameric compounds of K1 domains.

15. Process to obtain a multimeric compound comprising at least two K1 peptide domains as defined in claim 1, comprising the steps of:
synthesizing a molecule containing a K1 peptide domain linked to a biotin to obtain a biotinylated K1 molecule, said biotin being linked to the C-terminus of the K1 molecule,
mixing said biotinylated K1 molecule with a streptavidin homotetramer to obtain a composition of a multimeric compound comprising at least 2 K1 peptide domains,
purifying and separating multimeric compounds to obtain dimeric compounds of K1 domains, trimeric compounds of K1 domains, and tetrameric compounds of K1 domains.

16. Multimeric compound according to claim 2, which is a K1 dimer represented by the formula (II): 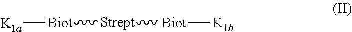
wherein:
K1ₐ and K1_{b} are polypeptides,
K1ₐ and K1_{b} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ and K1_{b} are C-terminally linked to a Biot by a covalent bond, and each Biot is linked to Strept by a non-covalent bond.

17. Multimeric compound according to claim 2, which is a K1 trimer represented by the formula (III): 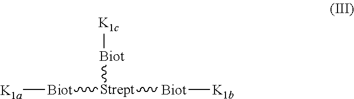
wherein:
K1ₐ, K1_{b} and K1_{c} are polypeptides,
K1ₐ, K1_{b} and K1_{c} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b} and K1_{c} are C-terminally linked to a Biot by a covalent bond, and each Biot is linked to the Strept by a non-covalent bond.

18. Multimeric compound according to claim 2, which is a K1 tetramer represented by the formula (IV): 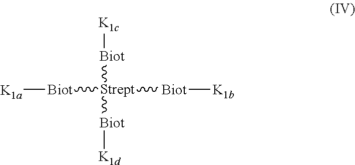
wherein:
K1ₐ, K1_{b}, K1_{c} and K1_{d} are polypeptides,
K1ₐ, K1_{b}, K1_{c} and K1_{d} contain a K1 peptide domain, said K1 peptide domain consisting of an amino acid sequence SEQ ID NO: 1 or of an amino acid sequence with at least 80%, preferably 90% identity to SEQ ID NO: 1,
Biot represents one molecule of biotin, and Strept represents one molecule of streptavidin,
K1ₐ, K1_{b}, K1_{c} and K1_{d} are C-terminally linked to a Biot by a covalent bond, and each Biot is linked to Strept by a non-covalent bond.

19. Multimeric compound according to claim 2, wherein K1ₐ and K1_{b}, and if present K1_{c} and K1_{d}, are identical.

20. Multimeric compound according to claim 3, wherein K1ₐ and K1_{b}, and if present K1_{c} and K1_{d}, are identical.


SEQUENCE LISTING (3 sequences)

SEQ ID NO: 1 - protein, 79 aa, Homo sapiens
CIIGKGRSYKGTVSITKSGIKCQPWSSMIPHEHSFLPSSYRGKDLQENYCRNPRGEEGGPWCFTSNPEVRYEVCDIPQC

SEQ ID NO: 2 - protein, 74 aa, Homo sapiens
CIIGKGRSYKGTVSITKSGIKCQPWSSMIPHEHSYRGKDLQENYCRNPRGEEGGPWCFTSNPEVRYEVCDIPQC

SEQ ID NO: 3 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS